(19) United States
(12) Patent Application Publication
(10) Pub. No.: US 2011/0027222 A1
(43) Pub. Date: Feb. 3, 2011

(54) Vaccine for the Treatment of Osteoarthritis

(76) Inventors: Wilhelmus Gerardus Johannes Degen, Boxmeer (NL); Virgil Elisabeth Joseph Caspar Schijns, Boxmeer (NL)

Correspondence Address: Intervet/Schering-Plough Animal Health, Patent Dept. K-6-1, 1990, 2000 Galloping Hill Road, Kenilworth, NJ, 07033-0530, US

(21) Appl. No.: 12/677,899

(22) PCT Filed: Sep. 24, 2008
(86) PCT No.: PCT/EP08/62722

§ 371 Date: Oct. 19, 2010

Related U.S. Application Data
(60) Provisional application No. 60/974,903, filed on Sep. 25, 2007.
(60) Provisional application No. 61/014,877, filed on Dec. 19, 2007.

(30) Foreign Application Priority Data
Sep. 25, 2007 (EP) 07117111.0
Dec. 20, 2007 (EP) 07150183.7

(51) Int. Cl. A61K 39/00; A61K 38/20; A61P 19/02

(52) U.S. Cl. 424/85.2

(57) ABSTRACT

The present invention pertains to a vaccine for the treatment of osteoarthritis in a vertebrate comprising IL-1β and optionally TNF-α cytokines or derivatives thereof, and in association with said IL-1β and optional TNF-α or derivatives thereof a part that is non-self with respect to the vertebrate such that the vaccine elicits an immune response in the vertebrate against IL-1β and TNF-α self-molecules of the said vertebrate.

The current invention pertains to a vaccine for the treatment of osteoarthritis in a vertebrate, the use of IL-1β and optionally TNF-α cytokines to produce such a vaccine and the treatment of osteoarthritis in a vertebrate by administering the vaccine.

Osteoarthritis (OA) is a non-inflammatory degenerative joint disease occurring chiefly in older humans and animals, which is characterized by degeneration of the articular cartilage, hypertrophy of bone at the margins and changes in the synovial membrane. Although this disease might arise from multiple origins, it is generally recognized that both mechanical and biochemical forces are leading causes of its appearance and progression. The disease is accompanied by pain and stiffness, particular after prolonged activity. It is a disease which is widespread under humans and animals, in particular dogs and horses, and as such is a serious issue in human as well as animal health.

Patients with mild OA may be treated only with pain relievers such as acetaminophen. Many patients however, are given nonsteroidal anti-inflammatory drugs (NSAID's). These NSAID's still only relieve the pain and have potentially dangerous side effects including inducing stomach ulcers, sensitivity to sun exposure, kidney disturbances, nervousness and depression. Some patients are treated with corticosteroids injected directly into the joints to slow the development of OA. This however is not preferred given the potential dangerous side effects of corticosteroids. In literature it is also suggested to treat OA by in situ treatment, i.e. in the OA joint, with antagonists for cytokines which are believed to play a role in mediating the increased matrix degradation that characterizes the OA cartilage lesion. This is for example known from Pelletier (Arthritis & Rheumatism, Vol. 40, No. 6, June 1997, pp 1012-1019) and Fernandes (Biorheology 39, 2002, 237-246) which describe a gene therapy treatment which is based on intra-articular injections of the interleukine receptor antagonist (IL-Ra) gene, which can reduce the progression of experimentally induced lesions. However, the positive effect is very short lived, and applicability as a treatment has yet to be proven. Also, the in situ administration of monoclonal antibodies directed against a cytokine itself, in particular interleukine 6, is known from European patent application EP 1 715 891 (Warner-Lambert Company, published in 2006). Disadvantages of this treatment are the high doses required which makes the treatment inherently expensive, the local (invasive) administration and the short-lived effect.

It is an object of the present invention to provide a treatment for osteoarthritis which does not, or at least to a lesser extent, suffers from the disadvantages of the known treatments for OA. Treatment in this sense may include treatment to prevent OA, provide relief for the clinical signs, mitigate or cure the disease or suppress progression of it after development has started.

To this end, a vaccine according to the preamble has been developed, comprising IL-1β and optionally TNF-α cytokines or derivatives thereof, and in association with said IL-1β and TNF-α or derivatives thereof a part that is non-self with respect to the vertebrate such that the vaccine elicits an immune response in the vertebrate against IL-1β and TNF-α self-molecules. Optionally the vaccine comprises a medium for carrying the IL-1β and TNF-α cytokines or derivatives thereof associated with the non-self part.

A vaccine in this respect is a constitution suitable for application to a vertebrate, i.e. any living animal having joints, such as fishes, amphibians, reptiles, birds and mammals, including humans (from now on the term “animal” is used to denote any vertebrate). In general a vaccine comprises one or more antigens such as attenuated or killed microorganisms and subunits thereof, or any other substance such as a metabolite of an organism. Upon administration of the vaccine to an animal, an immune response against the antigen(s) is elicited, which response should aid in preventing, ameliorating or treating a disease or disorder. Typically the antigen(s) are combined with a medium for carrying the antigens, often referred to as a “pharmaceutically acceptable carrier”. Such a carrier can be any solvent, dispersion medium, coating, antibacterial and antifungal agent, isotonic and absorption delaying agent, and the like that are physiologically compatible with the vertebrate. Some examples of such carrying media are water, saline, phosphate buffered saline, bacterium culture fluid, dextrose, glycerol, ethanol and the like, as well as combinations thereof. They may provide for a liquid, semi-solid and solid dosage form, depending on the intended mode of administration. As is commonly known, the presence of a carrying medium is not essential to the efficacy of a vaccine, but it may significantly simplify dosage and administration of the antigen.

A vaccine may additionally comprise non-specific immunostimulating agents, often referred to as adjuvants. In principal, each substance that is able to favor or amplify a particular process in the cascade of immunological events, ultimately leading to a better immunological response (i.e. the integrated bodily response to an antigen, in particular one mediated by lymphocytes and typically involving recognition of antigens by specific antibodies or previously sensitized lymphocytes), can be defined as an adjuvant. Note that the adjuvant is in general not required for the said particular process to occur, but favors or amplifies the said process.

The vaccine of the present invention is based on the use of IL-1β (interleukin 1β) and TNF-α (Tumor Necrosis Factor α) cytokines. Cytokines in general are non-antibody proteins released by a cell population on contact with antigens, which act as intercellular mediators in the generation of an immune response. Interleukine 1-β is a sub-group of the class of interleukines, a class of proteins that are secreted mostly by macrophages and T lymphocytes and induce growth and differentiation of lymphocytes and hematopoietic stem cells. It is a potent immuno-modulator which mediates a wide range of immune and inflammatory responses including the activation of B- and T-cells. TNF-α is the most well known member of the “Tumor Necrosis Factor” family, a family representing proteins produced i.a. by macrophages in the presence of an endotoxin and shown experimentally to be i.a. capable of attacking and destroying cancerous tumors. In the present case the vaccine comprises IL-1β and TNF-α or derivatives thereof and in association therewith a part that is non-self with respect to the vertebrate. Non-self in this sense means that it is immunogenic in the treated animal, i.e. it is capable of eliciting an immune response. As is commonly known, by using a non-self part in association with another molecule, an immune response can be provided against this other molecule, even if this other molecule is self with respect to the animal. A non self part (or multiple parts) in association with the cytokines could simply be provided for by choosing an IL-1β and TNF-α that is heterologous to the animal. Heterologous in this respect means not derived from the same species (as opposed to homologous). In this case, the cytokines inherently comprise in their molecular structure parts that are non-self with respect to the treated animal. Another possibility would for example be to choose a cytokine that is homologous to the treated animal, but is a mutant, or is provided with a foreign protein-part by recombinant techniques. Other ways of providing a non-self part in association with the cytokines or derivatives thereof are for example to physically or chemically couple one or more non-self molecules, a non-self structure, compound or just any non-self constitution to the cytokines. In any event, an immunogenic construct is provided wherein an operative connection exists between the cytokine or derivatives thereof and the non-self part, such that the construct is capable of eliciting an immune response against the self cytokines (i.c. IL-1β and TNF-α). As indicated here-above, the techniques for developing vaccines against self proteins (also known as autologous proteins) are commonly known since the mid eighties of the 20-th century, e.g. by chemical coupling of the self protein to a large foreign and immunogenic carrier protein (U.S. Pat. No. 4,161,519), or by preparation of fusion constructs between the self protein and the foreign carrier protein (WO 86/07383), or even by substituting as little as one single foreign T-helper epitope in the self protein (WO 95/05849). In these cases the non-self part of the immunogenic construct is responsible for the provision of epitopes for T-helper lymphocytes that render the breaking of autotolerance possible. It is noted that a derivative of IL-1β and/or TNF-α in respect of the present invention means a molecule which is smaller or larger than the starting cytokine, but still comprises one or more parts that are homologous to this starting cytokine, which part or parts constitute an antigenic determinant of these cytokines. Such part may be as small as one single epitope. Using as little as one single epitope has the advantage that very specific, in fact monoclonal antibodies, can be elicited against the self IL-1β and TNF-α. This reduces or even totally prevents the risk of cross reaction with other cytokines. This principle as such is commonly known and for example described in WO 2005/084198, WO 03/084979 and EP 218531, which explicitly disclose immunogenic peptides (i.e. peptides capable of inducing an immune response) of cytokines. It is noted that in the vaccine derivatives can be used for both cytokines at the same time, but also, that one of the cytokines is used in its natural form, and the other is a derivative of its natural form.

Applicant surprisingly found that by administering a vaccine according to the present invention, osteoarthritis can be successfully treated. In particular it has been found that when animals are vaccinated before they develop OA, less clinical signs appear when the animal actually develops OA. Clinical signs of the disease are suppressed without any severe chronic side-effects. Given these positive results, it leaves no doubt that contrary to what one would expect, adequate amounts of the elicited IL-1β and where applicable TNF-α antibodies reach the OA joint. Knowing that cytokine antagonists when present in an OA joint suppress OA progression (see Pelletier, Fernandes and Warner-Lambert Company as mentioned here-above), the animals treated with a vaccine according to the invention are less susceptible for progression of OA. Since OA is a progressive degenerative disease, it is understood that corresponding results are obtained when treating animals with the vaccine according to the invention after they have started developing OA. Without being bound to theory, the unexpected absence of severe chronic side-effects could be explained by assuming that vaccination with the vaccine according to the invention does not lead to a complete functional blockade of the self cytokines IL-1β and TNF-α in the animal, but to a reduction of the concentration of these cytokines, i.a. in the joint, to a normal level.

It is noted that Goldring (Clinical Orthopaedics and Related Research, No 427S, pp S27-S36) mentions that it is suggested that proinflammatory cytokines such as IL-1 and TNF-α contribute to the dysregulation of chondryte function that leads to the progressive degradation of the cartilage matrix and loss of joint function. However, he also states that a recent study by Clements (Arthritis & Rheumatism, Vol. 48, No. 12, pp 3452-3463, 2003) showed that gene deletion of IL-1β or IL-1β converting enzyme even accelerates the development of knee osteoarthritis! Thus, the prior art is ambiguous about the role of these cytokines in OA. It is not even known whether they unambiguously stimulate or suppress OA. This is acknowledged for example by a recent paper describing a research program to assess the role of cytokines in the development of OA (Baggio, Veterinary Immunology and Immunopathology 107 (2005) 27-39). This paper explicitly mentions that both IL-1 and TNF were reported to promote OA but that it is still open whether the abrogation of these cytokines is suitable to treat OA. Wildbaum (Immunity, Vol. 19, 679-688, November, 2003) even reports that neutralizing TNF-α suppresses the inflammatory disease rheumatoid arthritis but not osteoarthritis. Thus, the skilled practitioner would not unambiguously arrive at a IL-1β and optional TNF-α suppression when searching for a treatment for OA. Moreover, it is explicitly known from the prior art that where in situ treatment with a cytokine antagonists might help against OA, a systemic approach is unsuccessful (see EP 1 715 891; example 3). This is in line with the general understanding that the joints are fairly difficult to reach because of the blood-joint barrier (Bas et al., British Journal of Rheumatology, 1996; 35(6): 548-552 and Kushner et al., Arthritis Rheum, 1971; 14(5): 560-570).

It is also noted that many references are known that pertain to the treatment of acute inflammatory diseases, wherein it has been shown that the diseases can be treated by using a vaccine that comprises IL-1 and/or TNF-α to elicit an immune response against self IL-1 and/or TNF-α. Typical example of such a reference is WO 2007/039552, assigned to Cytos Biotechnology AG, Switzerland. In inflammation, as opposed to a disease such as OA, the role of cytokines is very clear: down regulation provides relief and is thus effective for treatment. In the said references it is often suggested or even stated, without disclosing proof in the form of examples, that the same treatment should also be effective in osteoarthritis because it is known that IL-1β and TNF-α may play a role in this disease. However, as explained here-above, OA is a non-inflammatory disease and therefore completely different from inflammatory diseases. The role of cytokines such as IL-1β and/or TNF-α is not yet clear and even seems to be ambiguous. Therefore, for the practitioner skilled in the field of Osteoarthritis, such references provide no justified base for even an expectation of a reasonable chance of success by treating OA using immunogenic IL-1β and/or TNF-α, let alone to expect that a immunogenic IL1β and optionally immunogenic TNF-α, given systemically, provides an adequate treatment of OA.

In short, there are two important reasons why the skilled man, based on the available knowledge about osteoarthritis, would refrain from developing a vaccine against IL-1β and optionally TNF-α to treat OA: Firstly, the prior art does not unambiguously teach what the role of cytokines, in particular IL-1β and TNF-α, in OA is and secondly, a systemic approach using a cytokine antagonist to regulate the role of cytokines in the joint itself has proven to be unsuccessful, even when in-situ treatment based on the same antagonist was proven to be successful. The skilled person therefore, based on the knowledge as is available today, would refrain from developing a vaccine against self-cytokines IL-1β and/or TNF-α to treat OA, even more so since this means that there will be a systemic attack against these cytokines which could be highly disadvantageous. Interleukin-1 for example is known to be involved in the immune response against microbial infection, to increase the number of bone marrow cells, to play a critical role in (oral) wound healing, to have antidiabetic effects and even to modulate cellular events during the late stages of pregnancy. A blockade of this cytokine is therefore commonly regarded undesirable.

In an embodiment, the IL-1β and TNF-α cytokines or derivatives thereof are homologous with respect to the treated vertebrate. Homologous in this sense means derived from the same species, as opposed to heterologous as defined here-above. Contrary to what one would expect, it has been found that antigen cytokines derived from the same species induce higher antibody titers in the treated vertebrate.

In another embodiment the part that is non-self with respect to the vertebrate comprises an antigenic determinant derived from a microorganism. A micro-organism in this sense means an organism of microscopic or submicroscopic size, in particular belonging to the bacteria, yeasts, molds, protozoa, algae, rickettsia, microbes or viruses. It appears that by including such an antigenic determinant, i.e. an epitope that provides an immunologically active region that binds to antigen-specific membrane receptors on lymphocytes or to secreted antibodies, high antibody titers can be provided against the self cytokines of the vertebrate.

In yet another embodiment, the part that is non-self with respect to the vertebrate comprises an adjuvant. As stated here-above, an adjuvant can in principle be any non-specific immunostimulating agent. The adjuvant part may be associated with the IL-1β and TNF-α cytokines or derivatives thereof by any chemical or physical bond, or in any other way as long as the adjuvant part is in operative connection with the IL-1β and TNF-α cytokines or derivatives thereof, i.e. as long as the adjuvant part is able to stimulate an immune response against these IL-1β and TNF-α cytokines or derivatives thereof, and thus also against corresponding antigenic determinants of the self IL-1β and TNF-α of the vertebrate. Adjuvants in general can be classified according to the immunological events they induce. The first class, comprising i.a. ISCOM's (immunostimulating complexes), saponins (or fractions and derivatives thereof such as Quil A), aluminum hydroxide, liposomes, cochleates, polylactic/glycolic acid, facilitates the antigen uptake, transport and presentation by APC's (antigen presenting cells). The second class, comprising i.a. oil emulsions, gels, polymer microspheres, non-ionic block copolymers and most probably also aluminum hydroxide, provide for a depot effect. The third class, comprising i.a. CpG-rich motifs, monophosphoryl lipid A, mycobacteria (muramyl dipeptide), yeast extracts, cholera toxin, is based on the recognition of conserved microbial structures, so called pathogen associated microbial patterns (PAMPs), defined as signal 0. The fourth class, comprising i.a. oil emulsion surface active agents, aluminum hydroxide, hypoxia, is based on stimulating the distinguishing capacity of the immune system between dangerous and harmless (which need not be the same as self and non-self). The fifth class, comprising i.a. cytokines, is based on upregulation of costimulatory molecules, signal 2, on APCs. An adjuvant helps in providing an adequate immune response. Therefore, it is less crucial that the non-self part of the cytokines or derivaties thereof is already capable of breaking autotolerance. An adjuvant therefore provides more freedom in the choice of antigens. In particular with adjuvants from class 1, embodied for example with saponins, fractions thereof and oil in water emulsions (e.g. liposomes), good results have been obtained.

In still another embodiment the IL-1β and TNF-α cytokines or derivatives thereof have a bioactivity that is reduced when compared with the self IL-1β and TNF-α cytokines of the vertebrate. A reduced bioactivity, i.e. a reduced effect of the agent upon the living animal or on its tissue, reduces the risk of acute side-effects upon administration of the vaccine. When the bioactivity of the cytokines is not reduced, acute side effects such as vomiting, shock, colic, etc. might occur. Reduction of bioactivity may be obtained by various cytokine-inactivation procedures such as the chemical formaldehyde treatment similar to that used for converting bacterial toxins into toxoids. Such methods are commonly known in the art of vaccine technology. In an alternative approach, mutant cytokines with a reduced bioactivity can be made. This is also commonly known in the art. It is noted that cytokines with a reduced bioactivity are also referred to as being bioinactive (although there might be substantial bioactivity left).

The invention also pertains to the use of IL-1β and optionally TNF-α cytokines to produce a vaccine for the treatment of osteoarthritis in a vertebrate and the treatment itself. The vaccine can be administered by any conventional route used in the art of vaccine technology, in particular by the intramuscular route, the sub-cutaneous, intra-dermal or sub-mucosal route or by the intravenous route, for example in the form of an injectable suspension. The administration can take place as a single dose or as a dose repeated one or more times after a certain interval. The suitable dose may vary inter alia as a function of the weight of the individual treated.

The invention will be explained in more detail by reference to the following examples.

### 1. Materials and Methods

### A. Induction of Antibodies Against self IL-1β and TNF-α in Dogs

Recombinant canine (Ca) and equine (Eq) proteins. CaIL-1β, CaTNF-α, EqIL-1β and EqTNF-α were cloned using standard Molecular Biological techniques using isolated RNA from peripheral blood lymphocytes (PBLs) extracted from dog or horse blood, respectively. The 3′-end of the Ca molecules were genetically fused to the minimal (17 aa) T-cell epitope from the Canine Distemper Virus fusion protein (CDV-F) (Ghosh et al. (2001) Immunology 104 pp. 58-66). The cDNA fragments encoding IL-1β and TNF-α were finally ligated in to a pET-vector such that the 5′-end was in-frame with a His-tag (for purification purposes). Equine IL-1β and TNF-α were not CDV-tagged. All proteins were expressed by E. coli, His-tag purified and checked for LPS content. The proteins used as antigens all had an LPS content<20 U/ml. SEQ ID 1 represents the DNA for the minimal CDV-F tagged Ca IL-1β. Nucleotides 1-75 represent the His-tag, nucleotides 532-585 represent the CDV F-epitope (17 amino acids+stop codon). SEQ ID 2 represents the minimal CDV-F tagged Ca IL-1β protein itself. SEQ ID 3 represents the DNA for the minimal CDV-F tagged Ca TNFα. Nucleotides 1-63 represent the His-tag, nucleotides 535-588 represent the CDV F-epitope (17 amino acids+stop codon). SEQ ID 4 represents the minimal CDV-F tagged Ca TNFα protein itself. SEQ ID 5 represents the DNA for the Equine IL-1β. Nucleotides 1-63 represent the His-tag. SEQ ID 6 represents the Equine IL-1β protein itself. SEQ ID 7 represents the DNA for the Equine TNFα. Nucleotides 1-63 represent the His-tag. SEQ ID 8 represents the Equine TNFα protein itself.

Next to these wildtype molecules as referred to in the paragraph above, a series of bio-inactive mutant (mt) molecules, to circumvent possible systemic adverse reactions, were generated using site-directed mutagenesis (note,: in this specification bioactive, wildtype molecules are referred to or indicated as “wt”; bio-inactive, or bio-“less”-active mutant molecules are referred to or indicated as “mt”; when no referral or indication is given, the wild-type form is meant). These mutants were genetically fused at the 3′-end to the minimal (17 aa) or maximal (32 aa) T-cell epitope from the Canine Distemper Virus fusion protein (CDV-F) (Ghosh et al. (2001) Immunology 104 pp. 58-66), and at the 5′-end (after the His-tag) to the maximal (35 aa) T-cell epitope from the Canine Parvo Virus (CPV) (Rimmelzwaan et al. (1990) J. Gen. Virology 71 pp. 2321-2329). Mutation sites were choosen based upon available scientific literature about human IL-1β and TNF-α: Simon et al. (1993) J. Biol. Chem. 268 pp.9771-9779 and Evans et al. (1995) J. Biol. Chem. 270 pp. 11477-11483 for IL-1β point mutations, and Zhang et al. (1992) J. Biol. Chem. 267 pp. 24069-24075 for TNF-α point mutations. One TNF-α mutant (mutant No 12, see below) is a derivative of specific point mutations combined with spontaneous mutations. All proteins were expressed by E. coli, His-tag purified and checked for LPS content. The tested proteins used as antigens all had an LPS content<20 U/ml. Eighteen bio-inactive mutants were made. The DNA that encodes for these mutants is listed below.

Mutant No 1 is encoded by the DNA for a minimal CDV-F tagged H30G CaIL-1β mutant. Nucleotides 1-75 of this DNA represent the His-tag, nucleotides 532-585 represent the minimal CDV-F epitope (17 amino acids+stop codon), nucleotides 163C>G and 164A>G represent the H30G mutation.

Mutant No 2 is encoded by the DNA for a minimal CDV-F tagged K92G CaIL-1β mutant. Nucleotides 1-72 represent the His-tag, nucleotides 529-582 represent the minimal CDV-F epitope (17 amino acids+stop codon), nucleotides 346A>G and 347A>G represent the K92G mutation.

Mutant No 3 is encoded by the DNA for a minimal CDV-F tagged H30G plus K92G CaIL-1β double mutant. Nucleotides 1-75 represent the His-tag, nucleotides 532-585 represent the minimal CDV-F epitope (17 amino acids+stop codon), nucleotides 163C>G and 164A>G represent the H30G mutation, nucleotides 349A>G and 350A>G represent the K92G mutation.

Mutant No 4 is encoded by the DNA for a minimal CDV-F tagged C7S CaIL-1R mutant. Nucleotides 1-75 represent the His-tag, nucleotides 532-585 represent the minimal CDV-F epitope (17 amino acids+stop codon), nucleotides 98G>C represent the C7S mutation.

Mutant No 5 is encoded by the DNA for a minimal CDV-F tagged K8E Ca IL-1β mutant. Nucleotides 1-75 represent the His-tag, nucleotides 532-585 represent the minimal CDV-F epitope (17 amino acids+stop codon), nucleotides 100A>G and 102G>A represent the K8E mutation.

Mutant No 6 is encoded by the DNA for a minimal CDV-F tagged L9S CaIL-1β mutant. Nucleotides 1-75 represent the His-tag, nucleotides 532-585 represent the minimal CDV-F epitope (17 amino acids+stop codon), nucleotides 104T>C represent the L9S mutation.

Mutant No 7 is encoded by the DNA for a minimal CDV-F tagged C7S plus K8E plus L9S Ca IL-1β triple mutant. Nucleotides 1-75 represent the His-tag, nucleotides 532-585 represent the minimal CDV-F epitope (17 amino acids+stop codon), nucleotides 98G>C represent the C7S mutation, nucleotides 100A>G and 102G>A represent the K8E mutation, nucleotides 104T>C represent the L9S mutation.

Mutant No 8 is encoded by the DNA for a maximal CPV and CDV-F tagged C7S CaIL-1β mutant. Nucleotides 1-75 represent the His-tag, nucleotides 76-171 represent the maximal CPV epitope (32 amino acids), nucleotides 628-726 represent the maximal CDV-F epitope (32 amino acids+stop codon), nucleotides 194G>C represent the C7S mutation.

Mutant No 9 is encoded by the DNA for a maximal CPV and CDV-F tagged K8E CaIL-1β mutant. Nucleotides 1-75 represent the His-tag, nucleotides 76-171 represents the maximal CPV epitope (32 amino acids), nucleotides 628-726 represent the maximal CDV-F epitope (32 amino acids+stop codon), nucleotides 196A>G and 198G>A represent the K8E mutation.

Mutant No 10 is encoded by the DNA for a maximal CPV and CDV-F tagged L9S CaIL-1β mutant. Nucleotides 1-75 represent the His-tag, nucleotides 76-171 represent the maximal CPV epitope (32 amino acids), nucleotides 628-726 represent the maximal CDV-F epitope (32 amino acids+stop codon), nucleotides 200T>C represent the L9S mutation.

Mutant No 11 is encoded by the DNA for a maximal CPV and CDV-F tagged C7S plus K8E plus L9S CaIL-1β triple mutant. Nucleotides 1-76 represent the His-tag, nucleotides 76-171 represent the maximal CPV epitope (32 amino acids), nucleotides 628-726 represent the maximal CDV-F epitope (32 amino acids+stop codon), nucleotides 194G>C represent the C7S mutation, nucleotides 196A>G and 198G>A represent the K8E mutation, nucleotides 200T>C represent the L9S mutation.

Mutant No 12 is encoded by the DNA for a maximal CPV and CDV-F tagged K8D plus L9S plus Q10del CaIL-1β triple mutant. Nucleotides 1-76 represent the His-tag, nucleotides 76-171 represent the maximal CPV epitope (32 amino acids), nucleotides 625-723 represent the maximal CDV-F epitope (32 amino acids+stop codon), nucleotides 196A>G and 198G>T represent the K8D mutation, nucleotides 200T>C represent the L9S mutation, compared to the wildtype sequence amino acid 10 was deleted (Q10del).

Mutant No 13 is encoded by the DNA for a minimal CDV-F tagged Y87S CaTNF-α mutant. Nucleotides 1-63 represent the His-tag, nucleotides 535-588 represent the minimal CDV-F epitope (17 amino acids+stop codon), nucleotides 323A>C represent the Y87S mutation.

Mutant No 14 is encoded by the DNA for a minimal CDV-F tagged Y119N CaTNF-α mutant. Nucleotides 1-63 represents the His-tag, nucleotides 535-588 represents the minimal CDV-F epitope (17 amino acids+stop codon), nucleotides 418T>A represent the Y119N mutation.

Mutant No 15 is encoded by the DNA for a minimal CDV-F tagged Y87S plus Y119N Ca TNF-α double mutant. Nucleotides 1-63 represents the His-tag, nucleotides 535-588 represent the minimal CDV-F epitope (17 amino acids+stop codon), nucleotides 323A>C represent the Y87S mutation, nucleotides 418T>A represent the Y119N mutation.

Mutant No 16 is encoded by the DNA for a maximal CPV and CDV-F tagged Y87S CaTNF-α mutant. Nucleotides 1-63 represent the His-tag, nucleotides 64-159 represent the maximal CPV epitope (32 amino acids), nucleotides 631-729 represent the maximal CDV-F epitope (32 amino acids+stop codon), nucleotides 419A>C represent the Y87S mutation.

Mutant No 17 is encoded by the DNA for a maximal CPV and CDV-F tagged Y119N CaTNF-α mutant. Nucleotides 1-63 represent the His-tag, nucleotides 64-159 represent the maximal CPV epitope (32 amino acids), nucleotides 631-726 represent the maximal CDV-F epitope (32 amino acids+stop codon), nucleotides 514T>A represent the Y119N mutation.

Mutant No 18 is encoded by the DNA for a maximal CPV and CDV-F tagged Y87S plus Y119N CaTNF-α double mutant. Nucleotides 1-63 represent the His-tag, nucleotides 64-159 represent the maximal CPV epitope (32 amino acids), nucleotides 631-726 represent the maximal CDV-F epitope (32 amino acids+stop codon), nucleotides 419A>C represent the Y87S mutation, nucleotides 514T>A represent the Y119N mutation.

Vaccine adjuvants. Used adjuvants are QuilA (250 μg/ml in 0.01 M phosphate buffered saline, abbreviated as PBS, also called “saline”), Matrix C (125 μg/ml in 0.01M PBS) and Microsol (25% Oil-in-Water emulsion). QuilA is a well-known saponin adjuvant isolated from the bark of the South American tree Quillaja saponaria Molina (Rosaceae family). It can be obtained from Biolang, KalveHave, Denmark or Roth, Karlsruhe, Germany. Matrix C (immunostimulating complex or ISCOM) is a vaccine adjuvant containing saponin, cholesterol and phospholipid (phophatidylcholine), which forms cage-like structures typically 40 nm in diameter. It can be obtained from CSL, Melbourne, Australia or Isconova, Uppsala, Sweden. Microsol is an oil-in-water emulsion consisting of small (typically below 1 μm) mineral oil droplets (Marcol 52 of ExxonMobil) in water, stabilized by using 1% Tween 80 detergent (polyoxyethylene (20) sorbitan monooleate), available from Acros Organics.

Experimental Design.

For the bioactive (wt) cytokines “only” experiments, the design is as follows. Five groups of approx. 4 month-old conventional beagle dogs were vaccinated s.c. in the right flank with 1.0 ml of the formulations as indicated in Table 1.

TABLE 1
Set up of the experimental vaccination in dogs.

| | N | Antigen | adjuvant |
|---|---|---|---|
| 1 | 4 | — | none: PBS |
| 2 | 5 | 50 μg Ca-His-IL-1β-CDV + | QuilA |
| | | 5 μg Ca-His-TNF-α-CDV | |
| 3 | 5 | 50 μg Ca-His-IL-1β-CDV + | Matrix C |
| | | 5 μg Ca-His-TNF-α-CDV | |
| 4 | 5 | 50 μg Ca-His-IL-1β-CDV + | Microsol |
| | | 5 μg Ca-His-TNF-α-CDV | |
| 5 | 5 | 50 μg Eq-N-His-IL-1β + | QuilA or PBS |
| | | 5 μg Eq-N-His-TNF-α | |

At 4, 8, 20 and 24 weeks after the first vaccination dogs received a booster vaccination of 1.0 ml (s.c. in the right flank). Bloodsamples were taken at T=0, 3, 6, 9, 12, 16, 20, 24, 28, 32 and 36 weeks after the first vaccination. Sera were used (1) to determine the antibody levels against CaIL-1β and CaTNF-α using antigen specific ELISAs; (2) for Western blot analysis; and (3) to check for neutralizing antibodies.

For the mixed bioactive (wt)/bioinactive (mt) cytokines experiments, the design is as follows. Six groups of 15-18 weeks-old conventional Beagle dogs were vaccinated s.c. in the right flank with 1.0 ml of the formulations as indicated in Table 1A. In this experiment 1 mutant CaIL-1β protein (mutant No 4: minimal CDV-F tagged C7S CaIL-1β mutant) and 1 mutant CaTNF-α protein (mutant No. 13: minimal CDV-F tagged Y87S Ca TNF-α mutant) were tested as single protein-antigen or in combination.

TABLE 1A
Set up of the experimental vaccination in dogs.

| | | | | i.a. NaCl |
|---|---|---|---|---|
| | | | | or urate |
| | N | Antigen | adjuvant | injection |
| 1 | 4 | — | none: PBS | NaCl |
| 2 | 4 | — | None: PBS | Urate |
| 3 | 4 | 50 μg wt Ca-His-IL- | Microsol | Urate |
| | | 1β-CDV-Fmin | | |
| 4 | 4 | 50 μg wt Ca-His-IL- | Microsol | Urate |
| | | 1β-CDV-Fmin | | |
| | | 5 μg wt Ca-His-TNF- | | |
| | | α-CDV-Fmin | | |
| 5 | 4 | 50 μg mt Ca-His-IL- | Microsol | Urate |
| | | 1β-C7S-CDV-Fmin | | |
| 6 | 4 | 50 μg mt Ca-His-IL- | Microsol | Urate |
| | | 1β-C7S-CDV-Fmin | | |
| | | 5 μg mt Ca-His-TNF- | | |
| | | α-Y87S-CDV-Fmin | | |

N-His = N-terminal His-tag;
wt = wildtype;
mt = mutant;
min = minimal;
i.a. = intra-articular

At 4, 8, 11, 14 and 16 weeks after the first vaccination dogs received a booster vaccination of 1.0 ml (s.c. in the right flank). Bloodsamples were taken at T=0, 4, 8, 11, 14, 16 and 17 weeks after the first vaccination. Sera were used to determine the antibody levels against CaIL-1β and CaTNF-α using antigen specific ELISAs (sandwich-catching approach).

ELISA and Western blot analysis. ELISA analyses were performed using standard procedures. For this, a catching polyclonal goat-anti-canine IL-1β and a catching monoclonal mouse-anti-canine TNF-α antibody were used to coat 96-well microtiter plates. C-terminally His (C-His)-tagged CaIL-1β or C-His-tagged CaTNF-α were than added to the plates subsequently followed by dilution series of dog sera. Binding of anti-IL-1β or anti-TNF-α specific antibodies were detected using a polyclonal rabbit-anti-canine IgG (H+L) HRP-labeled antibody.

Inhibition of IL-1β and TNF-α bioassay. An IL-1β and TNF-α responsive NIH-3T3 NFκB luciferase reporter cell line was used to measure inhibition. Pooled dog sera from several time points were pre-incubated with 10 ng/ml of Ca and Eq IL-1β and TNF-α proteins and tested for neutralizing, i.e. inhibition of receptor-binding, activity. Inhibition of IL-1β or TNF-α activity by antibodies was quantitated in Relative Light Units (RLU).

### B. Set Up of a Urate Crystal-Induced OA Model in Beagle Dogs

Experimental design. We used the urate crystals induced osteoarthritis model (see i.a. Bonneau et al; Revue Méd. Vét., 2005, 156, 4, 179-181). Two groups of 4 conventional beagle dogs (approx. 5 months-old) were intra-articularly injected into 1 knee joint (hind leg) with either 1.0 ml 0.9% NaCl (control group) or with 1.0 ml of 10 mg/ml urate crystals in 0.9% NaCl (OA model group). The intra-articular injections of 1.0 ml solution was performed under general anaesthesia. At T=0, 1 h, 2 h, 4 h, 6 h, 8 h, 24 h, 30 h, 48 h, 72 h and 96 h post-injection general behavior, lameness (scored at standing and walking), pain at palpation and knee joint effusion (swelling) were scored for each individual dog. At the end of the experiment the dogs were euthanized and the knees were macroscopically examined in the presence of a pathologist.

Urate crystals. A 10 mg/ml urate crystals (Sigma itemnr. U2875; batchnr. 120K5305) in 0.9% NaCl solution was used for intra-articular injection. For this, 22 g of urate crystals solution in 0.9% NaCl with a concentration of 50 mg/g was made. This solution was sonified until a suspension was obtained with particles ≦50 μm (microscopic examination). After the microscopic image confirmed that the particles had a size of 50 μm, 20 g suspension was diluted to 100 g with 0.9% NaCl (final concentration 10 mg/ml). The pH was adjusted to pH 7.0 and the suspension was autoclaved. The microscopic image of the suspension was also checked for crystal size (should be 50 μm) shortly before and after autoclaving. After preparation the suspension was stored at 2-8° C. until further use.

Identification. Dogs were numbered (tattooed) individually in the ear.

Housing. The dogs were housed individually in regular kennels under natural non-restricted circumstances, had outdoor exercise possibilities, and received water ad libitum. Crumbs were available to a limited extend.

TABLE 2
Grouping and dosing.

| gr. | N | test article |
|---|---|---|
| 1 | 4 | 1.0 ml 0.9% NaCl |
| 2 | 4 | 1.0 ml 10 mg/ml urate crystals in 0.9% NaCl |

Injection of 0.9% NaCl. The 4 animals from group 1 (control group) were intra-articularly injected with 1.0 ml 0.9% NaCl under general anesthesia into 1 knee joint (shaved left hind leg).

Injection of urate crystals. The 4 animals from group 2 (OA model group) were intra-articularly injected with 1.0 ml 10 mg/ml urate crystals in 0.9% NaCl under general aneasthesia into 1 knee joint (shaved left hind leg).

Experimental Procedures and Parameters.

Observation. At T=0, 1 h, 2 h, 4 h, 6 h, 8 h, 24 h, 30 h, 48 h, 72 h and 96 h post-injection general behavior, lameness (scored at standing and walking), pain at palpation and knee joint effusion (swelling) were scored for each individual dog. Dogs were first observed and scored in their cages, subsequently during walking and finally at standing on an observation table.

Lameness Scoring:

Standing Score

0: Stands normally with full weight-bearing.

1: Abnormal position with partial weight-bearing.

2: Abnormal position with non weight-bearing (3 legged dog).

3: Reluctant to rise.

Walking Score

0: Walks normally with full weight-bearing.

1: Slight lameness with partial weight-bearing.

2: Obvious lameness with intermittent partial weight-bearing.

3: Non weight-bearing (3 legged dog).

4: Reluctant to walk.

Trotting Score

0: Trots normally with full weight-bearing.

1: Slight lameness with partial weight-bearing.

2: Obvious lameness with intermittent partial weight-bearing.

3: Non weight-bearing (3 legged dog).

4: Reluctant to trot.

Pain at Palpation Score:

0: No signs of pain.

1: Mild to moderate pain (allows palpation but turning head or pulling away, vocalization, depressed).

2: Severe pain (does not allow examiner to palpate the joint).

Knee Joint Effusion (Swelling) in Comparison to the Non-Injected Joint Score:

0: No joint effusion (distinct patellar ligament palpable).

1: Mild effusion (minimal synovial filling with perceptible ligament).

2: Moderate effusion (obvious synovial filling with indistinct ligament).

3: Severe effusion (no ligament palpable).

### C. Set Up of a Urate Crystal-Induced OA Model in Shetland Ponies

Experimental design. We used the same osteoarthritis model in ponies. Two groups of 2 conventional Shetland ponies (approx. 12 months-old) were intra-articularly injected into 1 knee joint (right hind leg) with either 5.0 ml 0.9% NaCl (control group) or with 5.0 ml of 10 mg/ml urate crystals in 0.9% NaCl (OA model group). The intra-articular injections of 5.0 ml solution was performed under light sedation. At T=0, 1 h, 2 h, 4 h, 6 h, 8 h, 24 h, 30 h, 48 h, 72 h and 96 h post-injection general behavior, lameness (scored at standing and walking), pain at palpation and knee joint effusion (swelling) were scored for each individual pony. At the end of the experiment the ponies were euthanized and the knees were macroscopically examined by a pathologist.

Urate crystals. See section B.

Identification. The Shetland ponies were identified by an implanted chip and a numbered neckbelt.

Housing. The ponies, housed individually in conventional stable-boxes under natural non-restricted circumstances, received hay and water ad libitum, crumbs were available to limited extend.

TABLE 3
Grouping and dosing.

| gr. | N | test article |
|---|---|---|
| 1 | 2 | 5.0 ml 0.9% NaCl |
| 2 | 2 | 5.0 ml 10 mg/ml urate crystals in 0.9% NaCl |

Injection of 0.9% NaCl. The 2 animals from group 1 (control group) were intra-articularly injected with 5.0 ml 0.9% NaCl under light sedation into 1 knee joint (shaved right hind leg).

Injection of urate crystals. The 2 animals from group 2 (OA model group) were intra-articularly injected with 5.0 ml 10 mg/ml urate crystals in 0.9% NaCl under light sedation into 1 knee joint (shaved right hind leg).

Experimental Procedures and Parameters.

Observation. At T=0, 1 h, 2 h, 4 h, 6 h, 8 h, 24 h, 30 h, 48 h, 72 h and 96 h post-injection general behavior, lameness (scored at standing and walking), pain at palpation and knee joint effusion (swelling) were scored for each individual pony.

Ponies were observed and scored in their stable-boxes, and subsequently during walking at the corridor.

Lameness Scoring:

Standing Score

0: Stands normally with full weight-bearing.

1: Abnormal position with partial weight-bearing.

2: Abnormal position with non weight-bearing (3 legged pony).

3: Reluctant to rise.

Walking Score

0: Walks normally with full weight-bearing.

1: Slight lameness with partial weight-bearing.

2: Obvious lameness with intermittent partial weight-bearing.

3: Non weight-bearing (3 legged pony).

4: Reluctant to walk.

Pain at Palpation Score:

0: No signs of pain.

1: Mild to moderate pain (allows palpation but turning head or pulling away, vocalization, depressed).

2: Severe pain (does not allow examiner to palpate the joint).

Knee Joint Effusion (Swelling) in Comparison to the Non-Injected Joint Score:

0: No joint effusion (distinct patellar ligament palpable).

1: Mild effusion (minimal synovial filling with perceptible ligament).

2: Moderate effusion (obvious synovial filling with indistinct ligament).

3: Severe effusion (no ligament palpable).

### D. Prevention of OA Symptoms in Beagle Dogs Prophylactically Vaccinated Against IL-1β and TNF-α

Experimental vaccination design. In study A, for the “wt-only” experiments dogs were vaccinated at T=0, T=4, T=8, T=20 and T=24 weeks post vaccination with IL-1β and TNF-α formulated with several distinct immunopotentiators per group (see Table 1). For the present study these dogs were re-vaccinated twice 24 weeks after the last vaccination, i.e. at T=48 and T=54 weeks post-primo vaccination with 1.0 ml (s.c. in the right flank) vaccine formulation as indicated in Table 4. Blood samples for serology were taken at T=48, T=54 and T=58 weeks after the first vaccination. Sera were used to determine the antibody levels against IL-1β and TNF-α using antigen specific ELISAs and standard procedures.

TABLE 4
Set up of the experimental vaccination in dogs, continued from Table 1

| gr. | N | antigen | adjuvant | animal nr. |
|---|---|---|---|---|
| 1 | 6 | — | none: PBS | 223-381-384-834-892-924 |
| 2 | 4 | 50 μg Ca-His-IL-1β-CDV | QuilA | 8757-9140-8751-8759 |
| | | 5 μg Ca-His-TNF-α-CDV | | |
| 3 | 5 | 50 μg Ca-His-IL-1β-CDV | Microsol | 8749-9146-9126-8747-8761 |
| | | 5 μg Ca-His-TNF-α-CDV | | |
| 4 | 5 | 50 μg Eq-N-His-IL-1β | none: PBS | 8755-9134-8763-8753-9144 |
| | | 5 μg Eq-N-His-TNF-α | | |

For the mixed wt/mt experiments the experimental vaccination as depicted in Table 1A was used.

Induction of OA. For the wt-only experiments, one (1) week after the last blood samples were taken (T=59 weeks) dogs from group 1 (PBS control group) were split into 2 new groups (N=2 and N=4). All dogs from the 5 groups, approx. 18 months-old at this time point, were intra-articularly injected into 1 knee joint (hind leg) with either 1.0 ml 0.9% NaCl (control group 1) or with 1.0 ml of 10 mg/ml urate crystals in 0.9% NaCl (OA model groups 2-5). The intra-articular injections of 1.0 ml solution were performed under general anaesthesia. At T=1 h, 2 h, 4 h, 6 h, 8 h, 24 h, 32 h, 48 h, 72 h and 96 h post-injection general behavior, lameness (scored at standing and walking), pain at palpation and knee joint effusion (swelling) were scored for each individual dog. At the end of the experiment the dogs were euthanized and the knees were macroscopically examined in the presence of a pathologist.

TABLE 5
Set up up of the induction of OA in the vaccinated dogs.

| gr. | N | test article | animal nr. |
|---|---|---|---|
| 1 | 2 | 1.0 ml 0.9% NaCl | 223-381 |
| | | | (former group 1 of Table 4) |
| 2 | 4 | 1.0 ml 10 mg/ml urate crystals | 384-834-892-924 |
| | | in 0.9% NaCl | (former group 1 of Table 4) |
| 3 | 4 | 1.0 ml 10 mg/ml urate crystals | 8757-9140-8751-8759 |
| | | in 0.9% NaCl | (former group 2 of Table 4) |
| 4 | 5 | 1.0 ml 10 mg/ml urate crystals | 8749-9146-9126-8747-8761 |
| | | in 0.9% NaCl | (former group 3 of Table 4) |
| 5 | 5 | 1.0 ml 10 mg/ml urate crystals | 8755-9134-8763-8753-9144 |
| | | in 0.9% NaCl | (former group 4 of Table 4) |

For the mixed wt/mt experiments, the induction of OA was set up as indicated in Table 1A.

Lameness scoring: see section B.

Statistical analysis. Comparison of mean scoring values was performed using analysis of variance (ANOVA) with least significant difference (L.S.D.) as multiple comparison test. All results were generated using SAS Enterprise Guide 2 (SAS Institute Inc., Cary, N.C.) software. Differences were considered significant at a confidence level of 95% (P<0.05).

### 2. Results

### A. Induction of Antibodies Against Self IL-1β and TNF-α in Dogs.

Side effects resulting from vaccination (wt-only experiments). After primary vaccination (primo) local (skin) reactions were visible in dogs receiving the Eq proteins in QuilA. In the subsequent booster vaccinations we therefore replaced QuilA by PBS in this vaccination group (see also Table 1). Shortly after the first booster vaccination most of the dogs from groups 2 (Ca proteins in QuilA), 3 (Ca proteins in Matrix-C) and 4 (Ca proteins in Microsol) became ill, possibly because of the presence of a high concentration of (free) bioactive TNF-α in the vaccine formulation. These acute systemic side effects were prevented in subsequent booster vaccinations by reducing the amount of TNF-α, for both Ca and Eq proetins, from 50 μg/dose/dog to 5 μg/dose/dog.

Side effects resulting from vaccination (mixed wt/mt experiments). After primary vaccination (primo) local (skin) reactions were visible in most of the dogs receiving the Microsol adjuvant (groups 3-6). After the first and subsequent (booster) vaccinations local reactions were less prominent. No acute systemic side effects were observed at the used CaIL-1β and CaTNF-α protein concentrations.

Antibody titers against IL-1β and TNF-α (wt-only experiments). As depicted in FIGS. 1A-D significant ELISA antibody titers can be generated against the self-molecules CaIL-1β and CaTNF-α. It is clear from these figures that the antibodies are cross-reactive, i.e. the antibodies raised against the Ca proteins recognize the Eq proteins, and vice versa. It is remarkable that the antibodies raised against CaTNF-α recognize EqTNF-α at higher titer (possibly due to affinity/specificity) than the CaTNF-α protein, particularly at the early time points (FIGS. 1C and 1D). In general, higher overall antibody titers were generated in dogs vaccinated with the Ca proteins when compared to the corresponding Eq counterparts. Twelve weeks post-primo vaccination groups 1 and 3 were removed from the experiment due to housing limitations. In summary, it can be concluded that by using genetically modified canine self-molecules or heterologous equine proteins, it is possible to break immunological tolerance towards self and to induce high antibody titers. It is noted that with the mutant cytokines comparable results were obtained.

Western blot analysis. To confirm the specificity of the anti-IL-1β and anti-TNF-α antibodies Western blot analysis was performed using CDV-tagged and non-tagged proteins. Western blots were incubated with sera from T=9 weeks post primo-vaccination. As depicted in FIG. 2 all antibodies cross-react with the Ca and Eq IL-1β and TNF-α proteins. A non-related His-tag purified E. coli-expressed protein, chicken IL-18 (ChIL-18), was used as a control and was not recognized by the antibodies.

Neutralizing capacity of anti-IL-1β and anti-TNF-α antibodies. Although high antibody titers were induced in dogs against IL-1β and TNF-α, it was not clear whether these antibodies would be able to neutralize the bioactivity of the corresponding proteins. For this we used an IL-1β and TNF-α responsive NIH-3T3 NFκB luciferase reporter cell line. Ca and Eq IL-1β and TNF-α were pre-incubated with pooled sera from several time points and tested for neutralizing, i.e. inhibition of receptor binding, activity. From the results depicted in FIGS. 3A and 3B it can be concluded that pooled sera from 24 weeks post primo-vaccination contain the highest anti-CaIL-1β and anti-EqIL-1β neutralizing antibodies. Bioactivity, measured in Relative Light Units (RLU), of CaIL-1β and EqIL-1β is drammatically reduced when compared to the other sera. This is also the case, although less prominent, for inhibition of the bioactivity of CaTNF-α en EqTNF-α (FIGS. 3C and 3D). Pooled sera from 6 weeks and 9 plus 12 weeks post primo-vaccination contain few neutralizing IL-1β antibodies but significant neutralizing TNF-α antibodies.

Conclusion

In conclusion we have provided clear evidence that we can raise antibodies by active immunization against the minimal CDV-tagged wildtype and mutant CaIL-1β and CaTNF-α self molecules.

### B. Set Up of a Urate Crystal-Induced OA Model in Beagle Dogs

Injection of fluid into the knee joint. In general, the injection of 1.0 ml 0.9% NaCl or 1.0 ml 10 mg/ml urate crystals in 0.9% NaCl into the knee joint was performed without any serious side effects.

Clinical assessment: scoring of lameness. The animals injected with the urate crystals showed overt lameness, both at walking and standing, within 2 hours after injection (see FIG. 4). Two dogs recovered fully within 24 hours whereas the other 2 dogs suffered from the urate crystals until the end of the experiment. The results from the macroscopic examination of the knee joints of the euthanized dogs revealed that these 2 dogs had a patella luxation (anatomical joint abnormality). This patella luxation in combination with the injection of the urate crystals solution most probably resulted in the above mentioned prolonged period of discomfort. In the group of dogs injected with 0.9% NaCl solution only 1 dog suffered for a rather short period of time from the injection (visible at T=8 hr). Upon macroscopical examination of the knee joint of this dog it became clear that this animal also had a patella luxation.

Clinical assessment: pain at palpation and knee joint effusion (swelling). Pain at palpation and knee joint effusion were also monitored. As can be seen in FIG. 5 the SEMs (standard error of mean) are large indicating that there is quite some variation between individual dogs. Between 2 and 8 hours post injection of urate crystals pain at palpation is measurable with a maximum between 4 and 6 hours. Knee joint effusion is somewhat delayed compared to pain at palpation and starts at 4 hours post injection with a maximum at 8 hours.

Clinical assessment: knee pathology. At the end of the experiment all dogs were euthanized and the knees were examined macroscopically. In all dogs no traces of injection solution were found. Only in 1 dog the injection site was still visible. In the knees of 3 dogs a large amount of fluid was visible. It proved that these 3 dogs had a patella luxation.

Conclusion

The applied arthritis model in dogs by intra-articular injection of a solution containing urate crystals into 1 knee joint (hind leg) is rapid (within 2 hours effects are visible), short-lived (hours to 1-2 days) and reversible. Relevant parameters to monitor the process include scoring of lameness at walking and standing, pain at palpation and joint effusion. Body temperature and knee temperature are no relevant parameters in this model.

### C. Set Up of a Urate Crystal-Induced OA Model in Shetland Ponies

Injection of fluid into the knee joint. In general, the injection of 5.0 ml 0.9% NaCl or 5.0 ml 10 mg/ml urate crystals in 0.9% NaCl into the knee joint was performed without any serious side effects.

Clinical assessment: scoring of lameness. The ponies injected with the urate crystals showed overt lameness, both at walking and standing, within 2 hours after injection (see FIG. 6). Maximum discomfort was scored between 6 and 8 hours post-injection. The 2 control ponies showed no signs of lameness.

Clinical assessment: pain at palpation and knee joint effusion (swelling). Pain at palpation and knee joint effusion were also monitored. As can be seen in FIG. 7A pain was scored within 2 hours post-injection whereas knee joint effusion (FIG. 7B) was detectable at a later stage (>48 hours). As the knee joint effusion does not increase between 72 and 96 hours, this might indicate that the swelling reaches a maximum at 96 hours.

Clinical assessment: knee pathology. At the end of the experiment all ponies were euthanized and the knees were macroscopically examined. In all ponies no traces of injection solution could be found. Macroscopical examination of the knee joints of the ponies injected with urate showed an increased amount of yellow synovial fluid, thickening of the synovial membrane with oedema and haemorrhage. The controls showed a no pathology.

Conclusion

The applied arthritis model in ponies by intra-articular injection of a solution containing urate crystals into 1 knee joint (right hind leg) is, as we also showed in dogs, rapid (within 2 hours effects are visible), short-lived (hours to 1-2 days) and reversible when scoring lameness and pain. Knee joint effusion (swelling) is palpable starting at 48 hours post-injection and appears to reach a maximum between 72 and 96 hours.

### D. Prevention of OA Symptoms in Beagle Dogs Prophylactically Vaccinated Against IL-1β and TNF-α

Antibody titers against IL-1β and TNF-α after re-vaccination. As depicted in FIGS. 8A-D (wt-only experiments) the overall antibody titers at T=48 weeks post-vaccination, i.e. 24 weeks post-last booster vaccination, were maintained at a rather high level. Re-vaccination of the dogs resulted in a moderate increase in antibody titer across all tested antigens and all groups.

Clinical assessment: discomfort score. In FIG. 9 (wt-only experiments) the discomfort scores for ‘standing’ (FIG. 9B), ‘walking’ (FIG. 9C), ‘pain at palpation’ (FIG. 9D), ‘knee joint effusion (swelling)’ (FIG. 9E) and the ‘total clinical score’, which is the average of all discomfort scores measured, (FIG. 9A) are depicted. From all figures it is clear that non-vaccinated dogs showed overt discomfort within 2 hours after injection. The vaccinated dogs, showed, in several cases significantly, reduced or moderate to mild discomfort. As can be seen in the figures the discomfort scores for the vaccinated dogs were significantly lower and showed a delay when compared to the non-vaccinated dogs.

Clinical assessment: knee pathology. At the end of the experiment all dogs were euthanized and the knees were macroscopically examined by a pathologist. In general, except for the NaCl control dogs, in all dogs the synovial membrane appeared to be thickened and reddish. Next to this in all knees a small amount of mucous fluid was detected.

Clinical assessment: discomfort score. In FIG. 10 (mixed wt/mt experiments) the discomfort scores for ‘standing’ (FIG. 10A) and ‘walking’ (FIG. 10B) are depicted at the time points 6 hours and 8 hours post intra-articular urate injection. From both figures it is clear that non-vaccinated dogs (urate control group) showed overt discomfort at 6 hours and 8 hours after injection. The vaccinated dogs, showed, in several cases significantly, reduced or moderate to mild discomfort. As can be seen in FIG. 10 the discomfort scores for the vaccinated dogs were significantly lower when compared to the non-vaccinated dogs (urate control group). Also clear at, especially, 8 hours post-injection is that vaccination with only wildtype CaIL-1β or only mutant CaIL-1β reduces discomfort when compared to non-vaccinated urate-control dogs.

Conclusion

From the results obtained from this experiment we can conclude that it is possible to induce antibodies against the self-molecules IL-1β and TNF-α, based on the use of either wildtype or mutant cytokines, and that these antibodies, directed either against IL-1β alone or also directed against TNF-α, are able to suppress arthritis symptoms. It can also be concluded that the best results seem to be obtained with a vaccine that is directed against both IL-1β and TNF-α.

### E. Various

It is noted that with using bioactive IL-1β and TNF-α, we have seen acute side effects. These side-effects can be prevented by using bio-inactive versions of these cytokines, for example as known from U.S. Pat. No. 6,093,405. One could choose for partially inactivated cytokines to obtain a balance between immunogenicity and bioactivity. We used the urate crystals model for inducing OA. It is noted that other models for OA are known from the prior art, for example the Anterior Cruciate Ligament Transection model (ACLT, described in Fleming et al; Curr Opin Orthop. 2005 October; 16(5): 354-362) or the Groove model (described in Mastbergen et al; Rheumathology, 2006; 45(4): 405-413). Given the positive results of the experiments as described in the present application, it is believed that corresponding results will be obtained when applying these models or patients suffering from or developing OA due to natural causes. We have shown a significant effect in the relief for OA patients when vaccinated with a combination of IL-1β and TNF-α or derivatives thereof. This has been shown in beagle dogs. Given the results in this vertebrate, and the resemblance in physiological processes pertaining to joints in the group of vertebrates, in particular mammalian vertebrates such as dogs, horses and humans, the current invention can be used in any vertebrate, in particular mammalian vertebrate.

### F. DESCRIPTION OF THE FIGURES

FIG. 1 shows IL-β and TNF-α specific antibody responses measured at several time points post-vaccination. Dogs were either not-immunized (saline control), immunized with [CaIL-1β+CaTNF-α] proteins (abbreviated with Ca) formulated in QuilA, Matrix C, or Microsol, or immunized with [EqIL-1β+EqTNF-α] proteins (abbreviated with Eq) formulated in QuilA (primo only)/saline (boosters). At 4, 8, 20 and 24 weeks after the primo-vaccination dogs received a booster vaccination. Antibodies were measured using antigen specific ELISAs in which proteins were used without CDV-tag. [A]. antibody titers measured against CaIL-1β; [B]. antibody titers measured against EqIL-1β; [C]. antibody titers measured against CaTNF-α; [D]. antibody titers measured against EqTNF-α. T=weeks post primo-vaccination.

FIG. 2 shows a Western blot analysis of CDV-tagged and un-tagged CaIL-[3, CaTNF-α, and EqIL-1β and EqTNF-α proteins. Proteins, 1 μg/lane, were analyzed by 4-12% Nu-PAGE and Western blotting using the sera from 1 dog from each vaccination group at 9 weeks post primo-vaccination. [A]. Coomassie stained gel; [B]. Western blot incubated with control serum; [C]. Western blot incubated with serum from a dog vaccinated with [CaIL-1β+CaTNF-α] formulated in QuilA adjuvant; [D]. Western blot incubated with serum from a dog vaccinated with [CaIL-1β+CaTNF-α] formulated in Matrix C adjuvant; [E]. Western blot incubated with serum from a dog vaccinated with [CaIL-1β+CaTNF-α] formulated in Microsol adjuvant; [F]. Western blot incubated with serum from a dog vaccinated with [EqIL-1β+EqaTNF-α] formulated in QuilA (primo only)/saline (boosters) adjuvant.

FIG. 3 shows the inhibition of IL-1β- or TNF-α-induced NFκB activation by sera from vaccinated dogs. Ten ng/ml of [A] CaIL-1β, [B] EqIL-1β, [C] CaTNF-α, or [D] EqTNF-α were mixed with dilutions of antibody sera from dogs vaccinated with [CaIL-1β+CaTNF-α] and incubated with NIH-3T3 reporter cells. Inhibition of IL-1β or TNF-α activity by antibodies was quantitated in Relative Light Units (RLU). T=6 weeks: this is pooled sera from dogs of groups 2+3+4 (see Table 1) taken 6 weeks post primo-vaccination. T=9+12 weeks: this is pooled sera from dogs of groups 2+3+4 (see Table 1) taken 9 and 12 weeks post primo-vaccination. T=24 weeks: this is pooled sera from dogs of groups 2+4 (see Table 1) taken 24 weeks post primo-vaccination.

FIG. 4 shows mean scores at standing [A] and walking [B] and mean total lameness scores [C] (standing+walking) for beagle dogs. Data are expressed as geometric mean±SEM.

FIG. 5 shows mean scores of pain at palpation [A] and mean scores of knee joint effusion [B] for beagle dogs. Data are expressed as geometric mean±SEM.

FIG. 6 shows mean scores at standing [A] and walking [B] and mean total lameness scores [C] (standing+walking) for Shetland ponies. Data are expressed as geometric mean±SEM. The arrow at the left vertical axis indicates the maximal score for the indicated parameter.

FIG. 7 shows the mean scores for pain at palpation [A] and the mean scores for knee joint effusion [B]. FIG. 7 [C] shows the total clinical score (standing+walking+pain+swelling) for Shetland ponies. Data are expressed as geometric mean±SEM. The arrow at the left vertical axis indicates the maximal score for the indicated parameter.

FIG. 8 shows IL-β and TNF-α specific antibody responses measured at several time points post-primo vaccination. Dogs were either not-vaccinated (saline control) or re-vaccinated at T=48 and T=54 weeks post-primo vaccination with [CDV-tagged CaIL-1β+CaTNF-α] proteins (abbreviated with Ca) formulated in QuilA or Microsol, or re-vaccinated with [EqIL-1β+EqTNF-α] proteins (abbreviated with Eq) formulated in QuilA (primo only) or saline (boosters). Antibodies were measured at T=48, T=54 and T=58 weeks post-primo vaccination using antigen specific ELISAs in which proteins were used without CDV-tag. [A] antibody titers measured against CaIL-1β; [B] antibody titers measured against EqIL-1β; [C] antibody titers measured against CaTNF-α; [D] antibody titers measured against EqTNF-α. ★=significantly different (P<0.05) from saline group; [symbol] =significantly different (P<0.05) between indicated groups. T=weeks post primo-vaccination.

FIG. 9 shows mean total clinical score for beagle dogs. FIG. 9 [A] gives the total clinical score (standing+walking+pain at palpation+knee joint swelling). FIG. 9 [B] gives the mean score at standing. Walking is shown in [C] and pain at palpation in [D], knee joint effusion (swelling) in [E]. Data are expressed as geometric mean±SEM.

★=significantly different (P<0.05) from urate crystal control group at the indicated time point. [symbol] =significantly different (P<0.05) between indicated groups.

FIG. 10 shows mean clinical score for beagle dogs. [A]. mean clinical score at standing; [B]. mean clinical score at walking. Data are expressed as geometric mean±SEM. ★=significantly different (P<0.05) from urate crystal control group at the indicated time point.

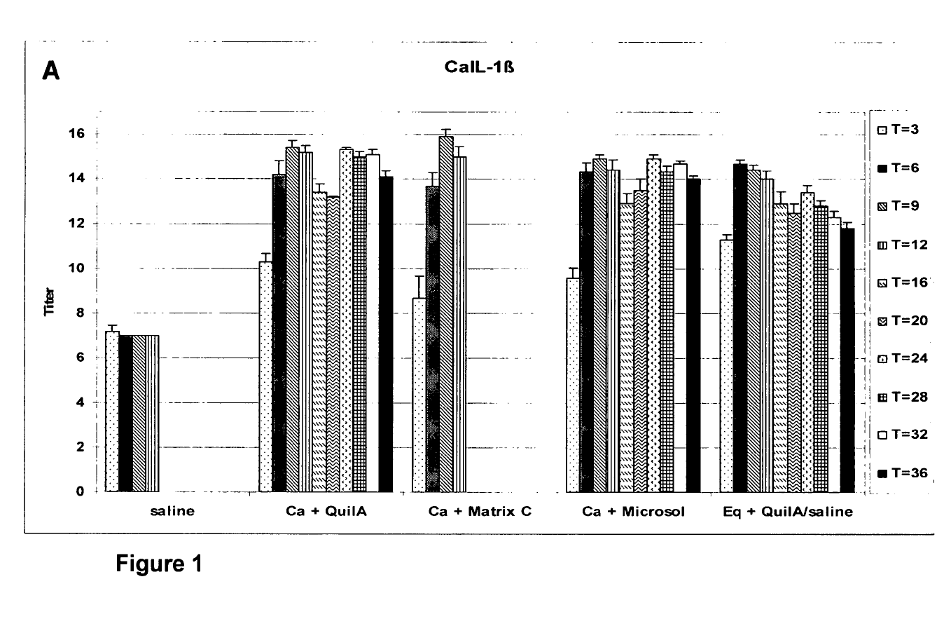

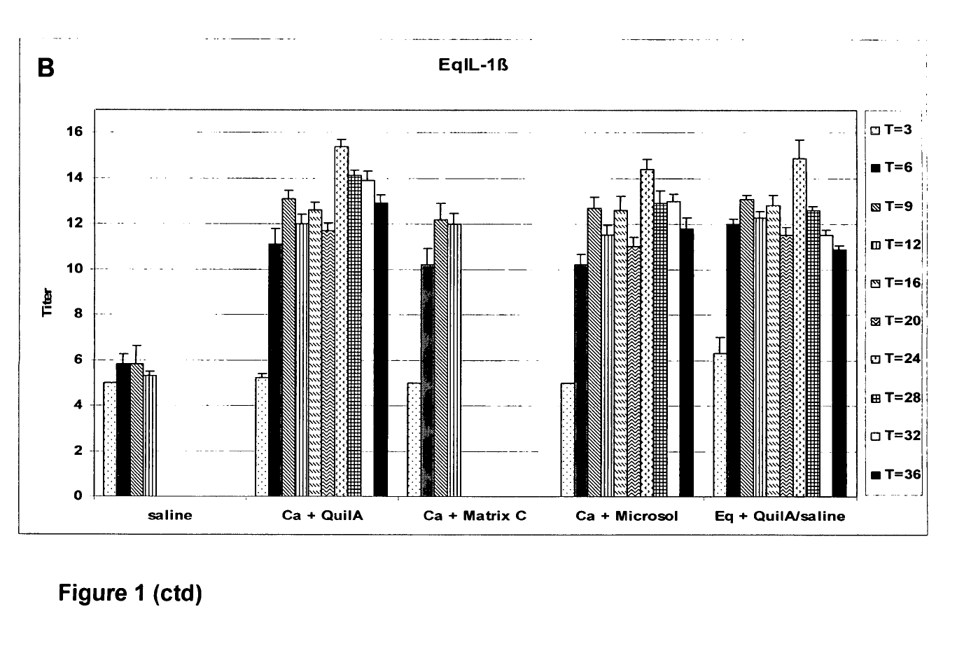

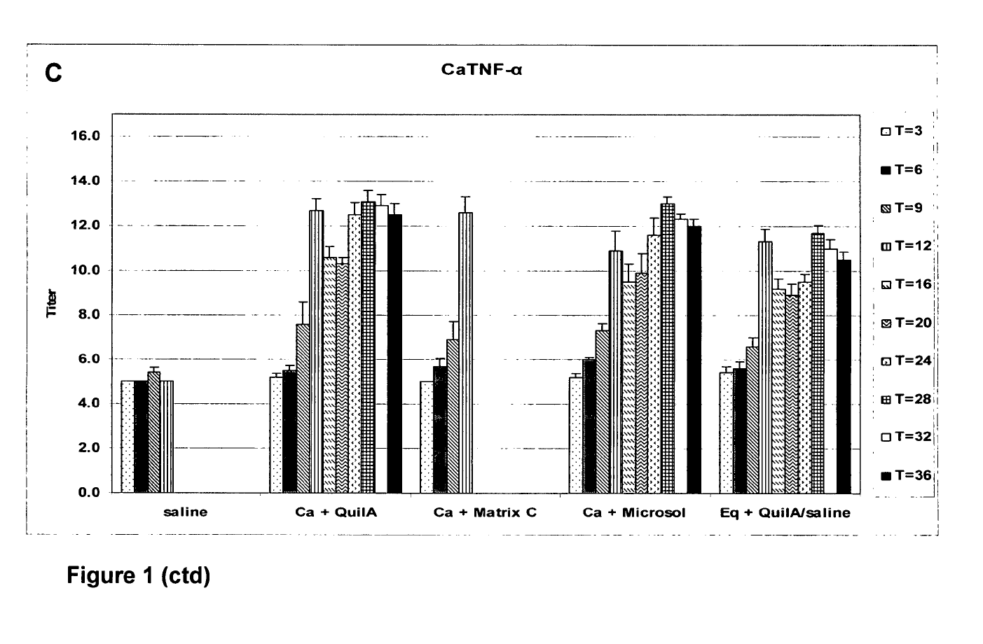

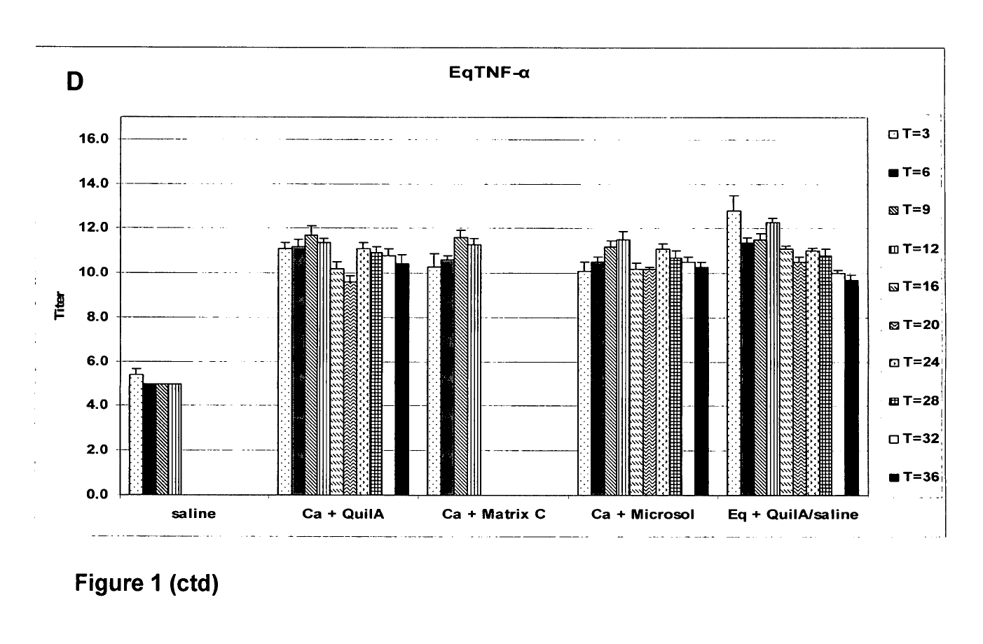

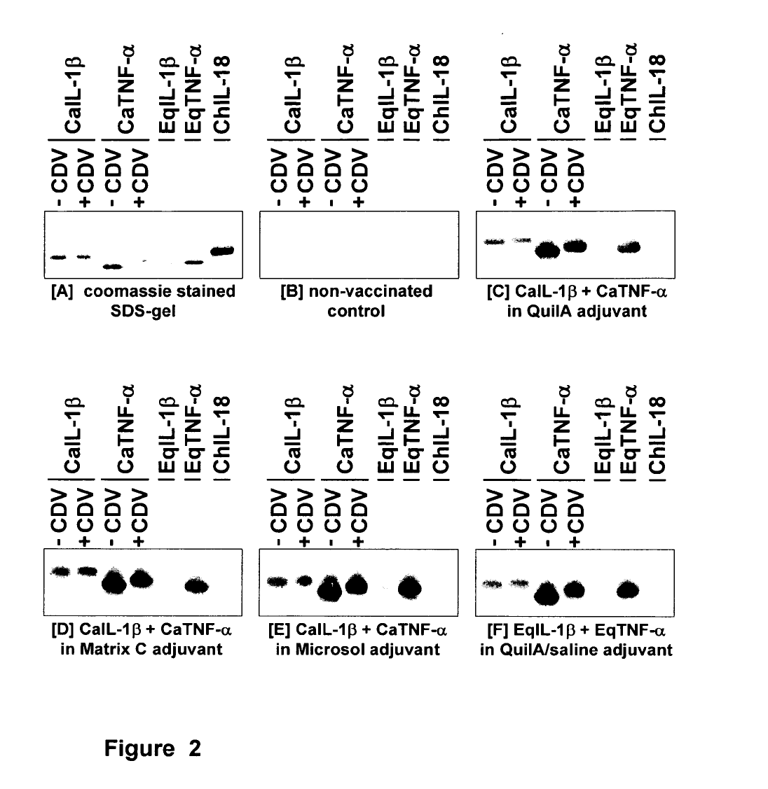

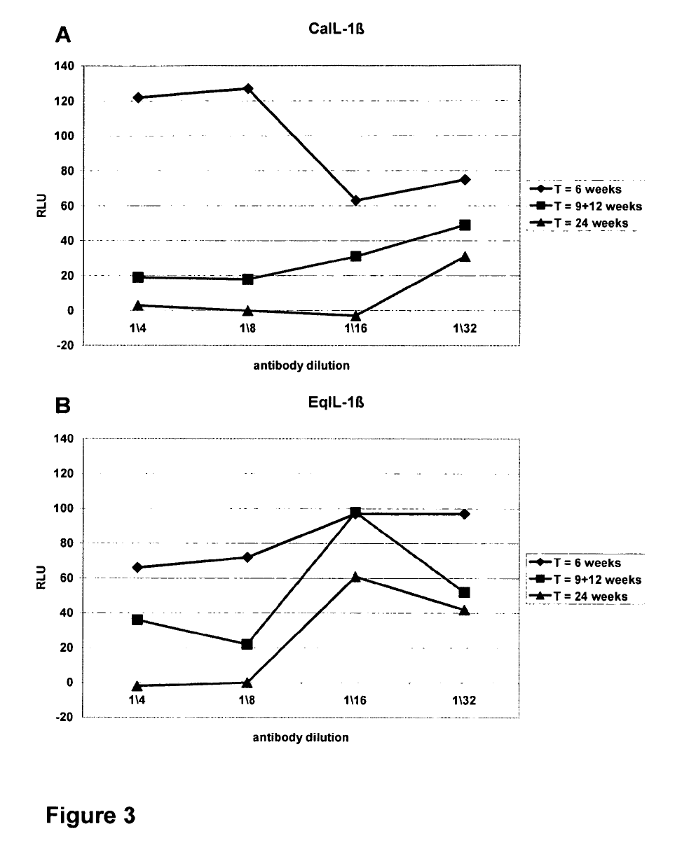

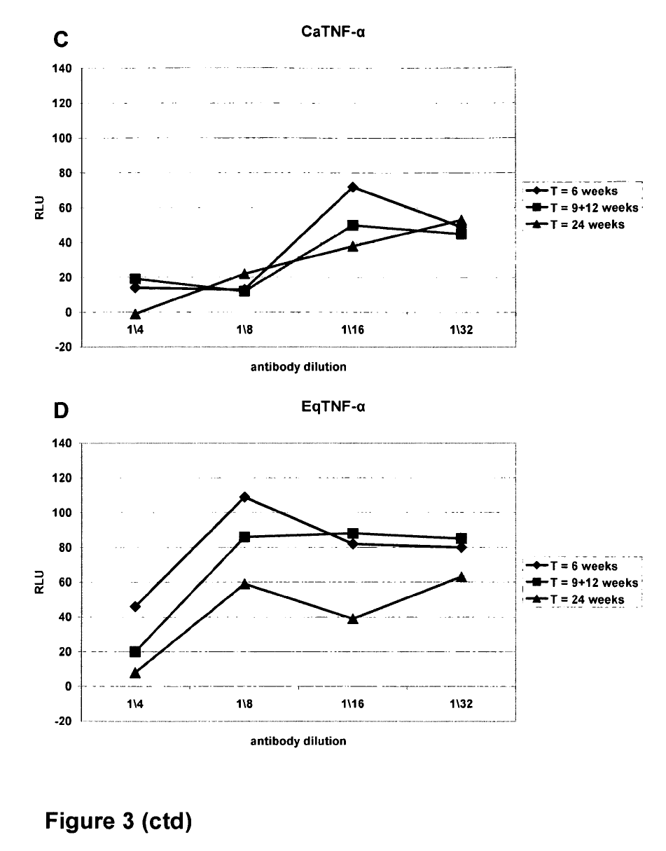

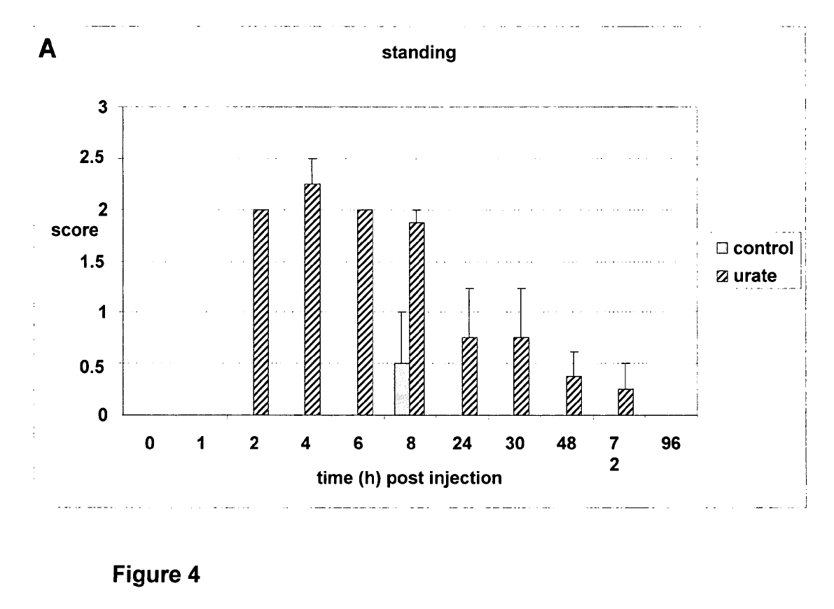

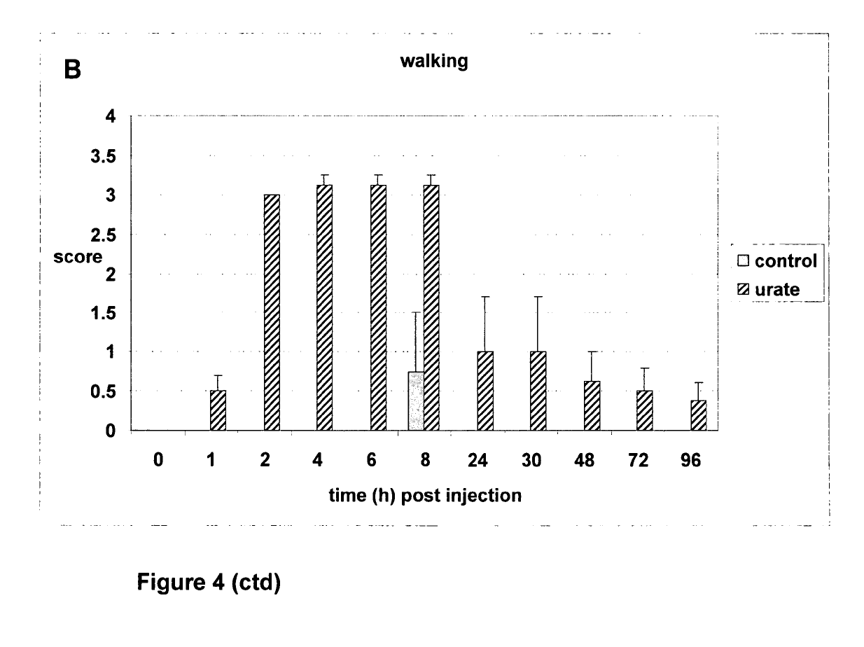

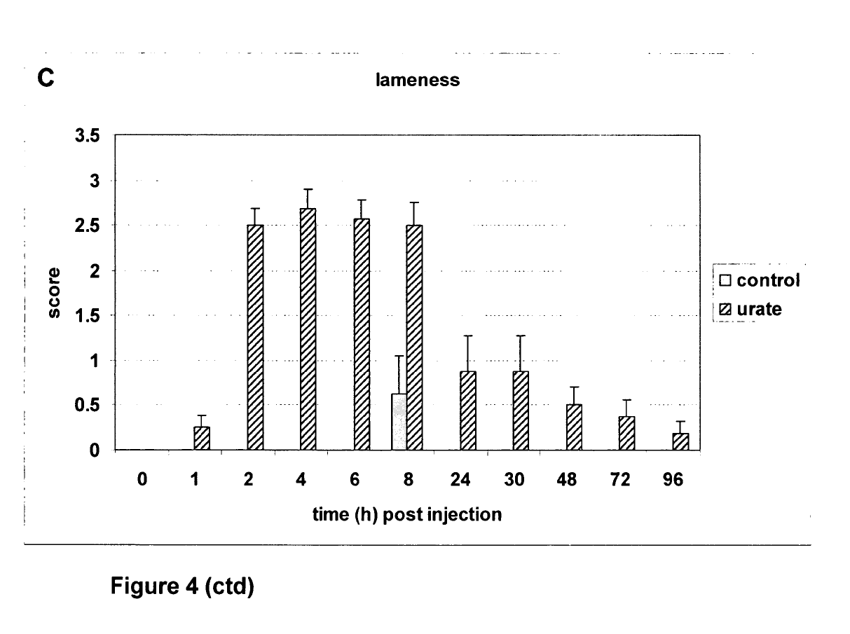

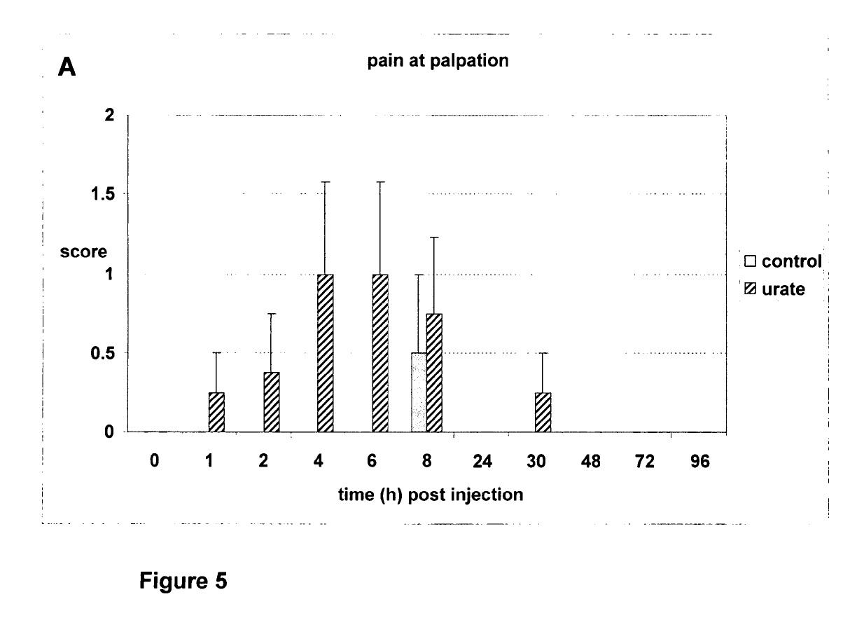

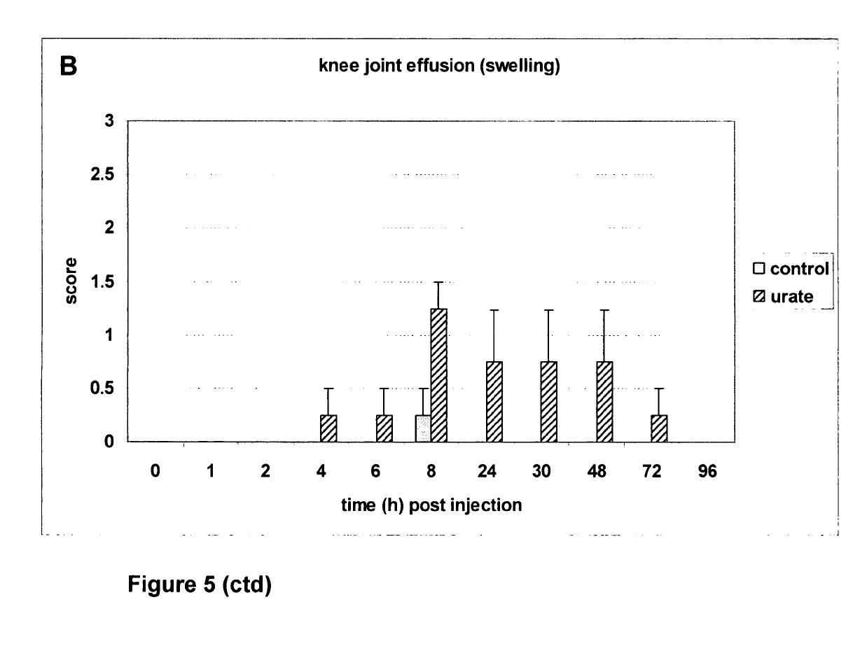

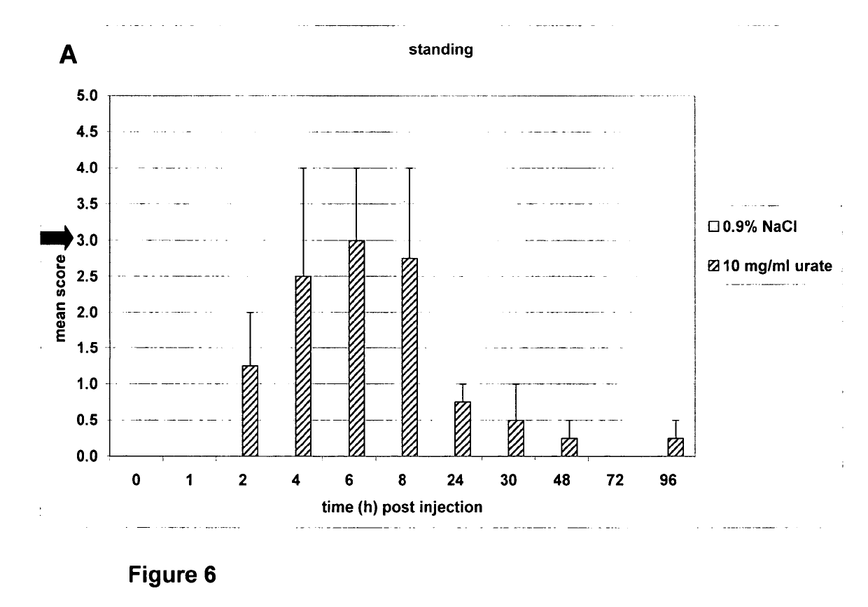

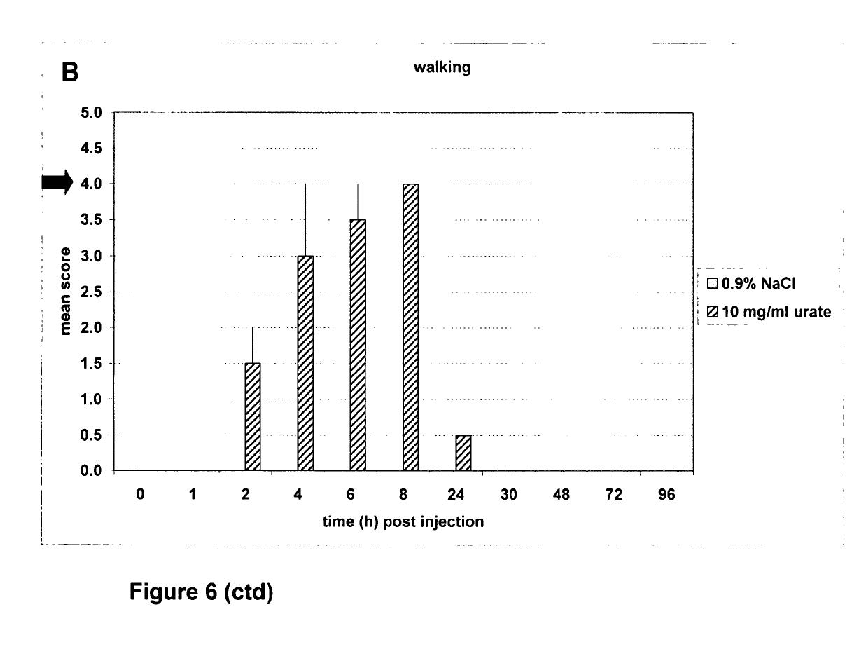

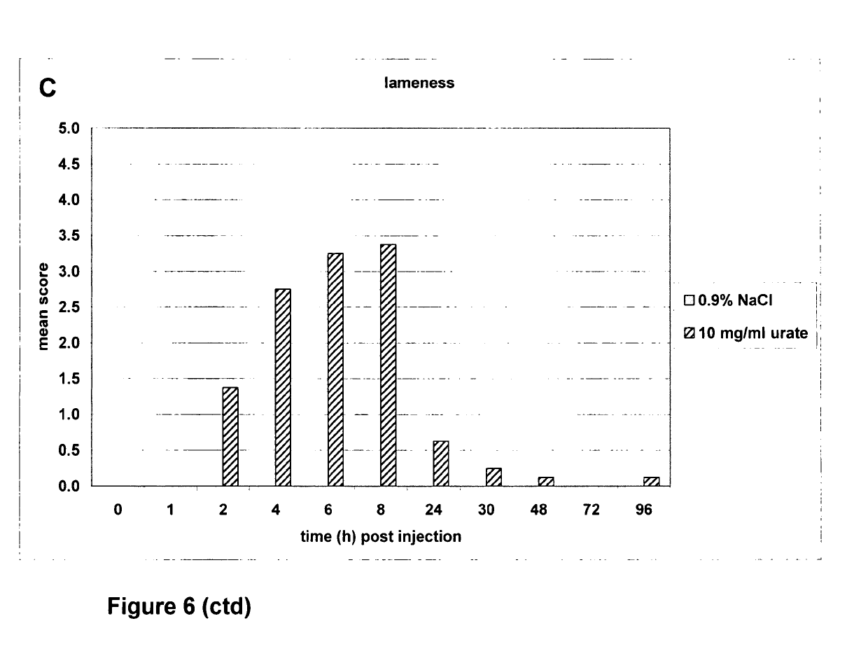

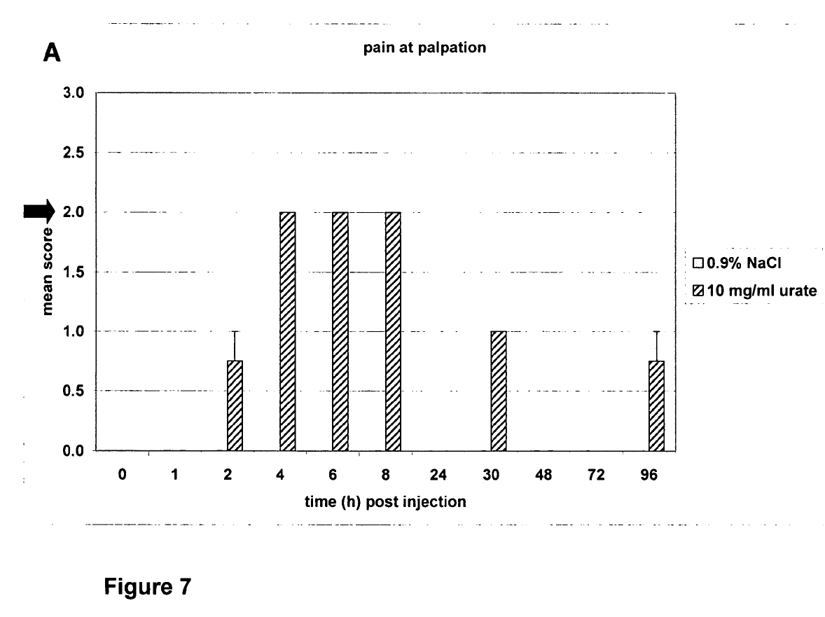

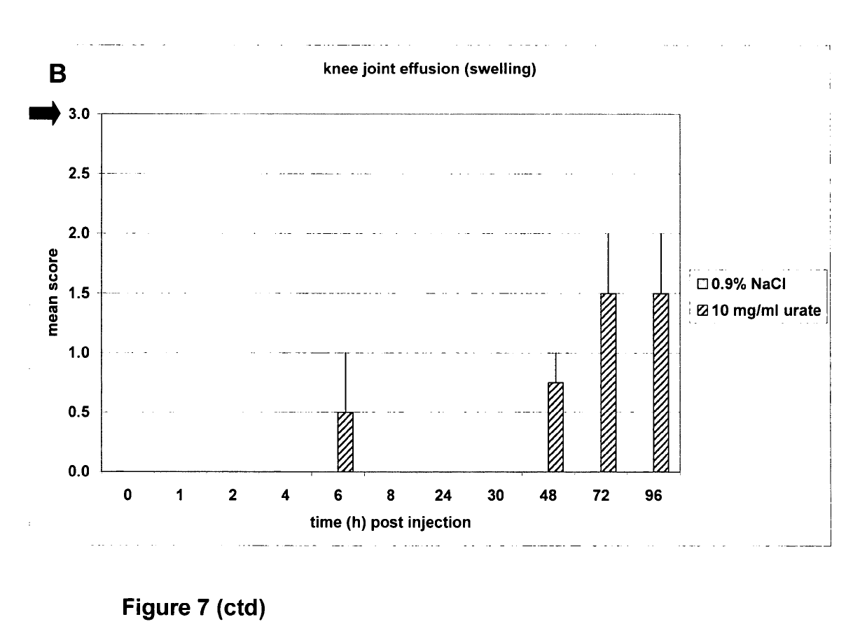

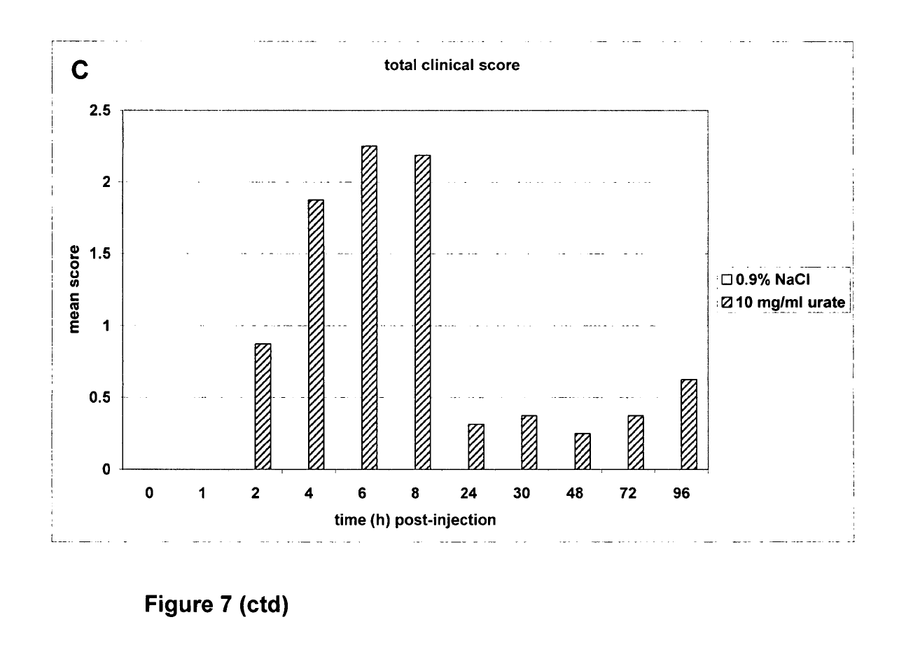

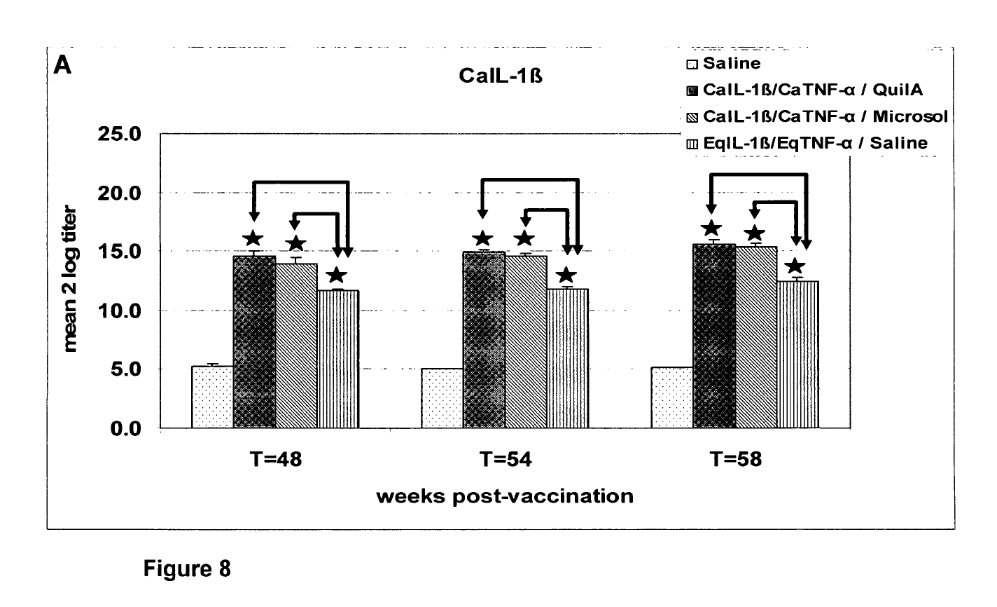

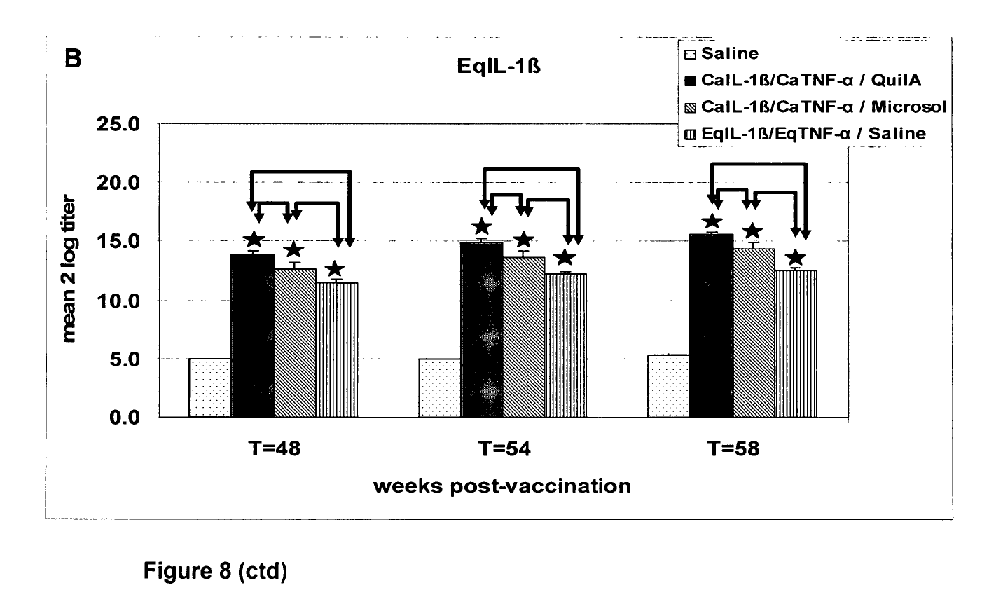

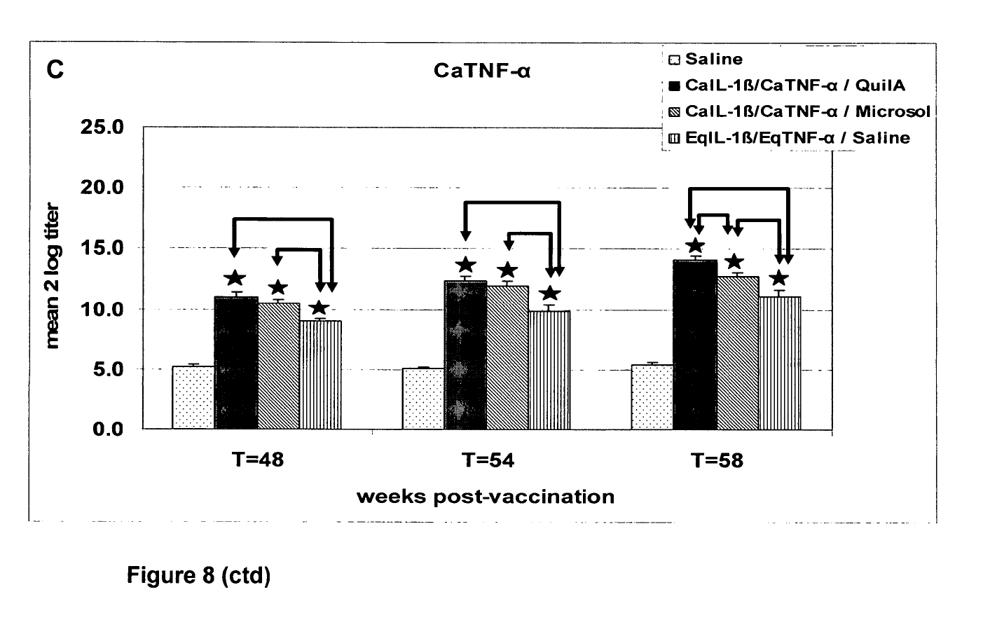

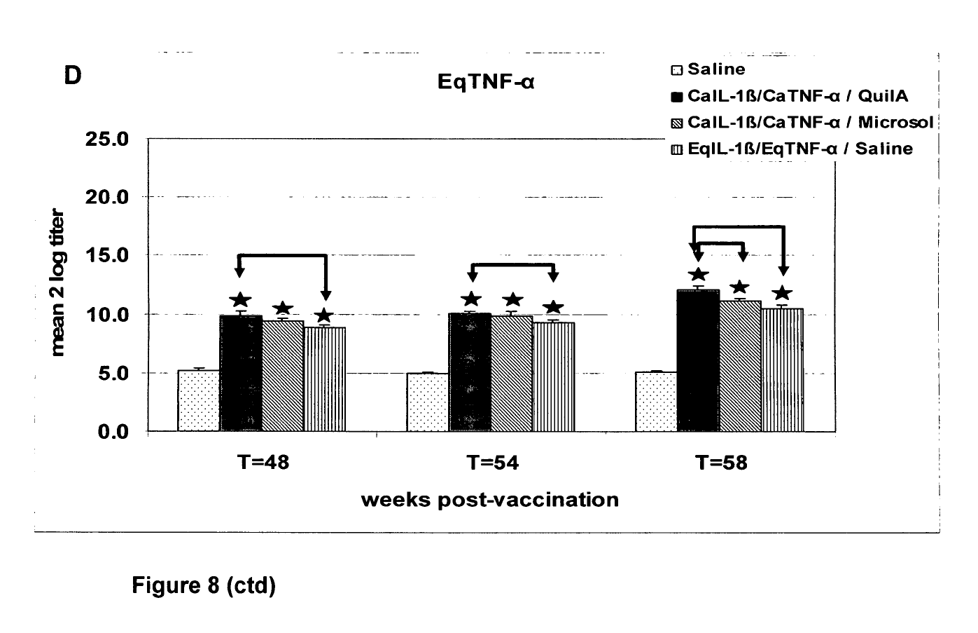

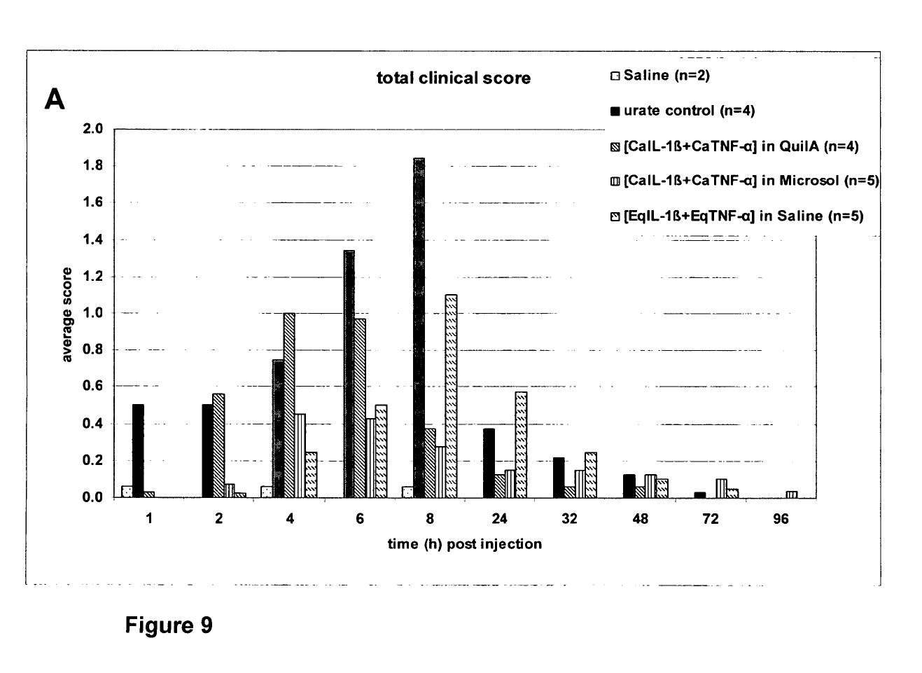

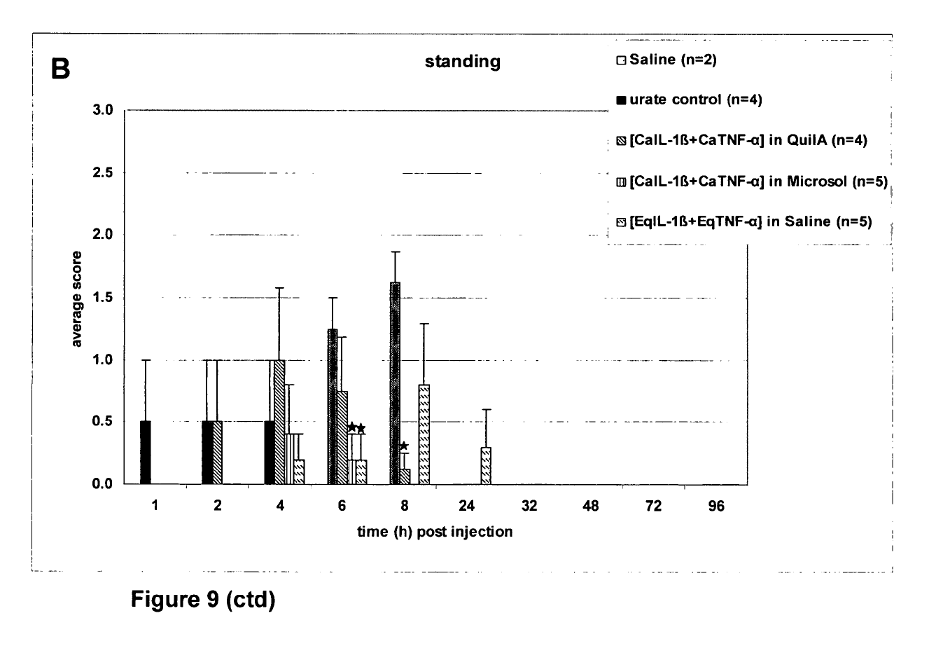

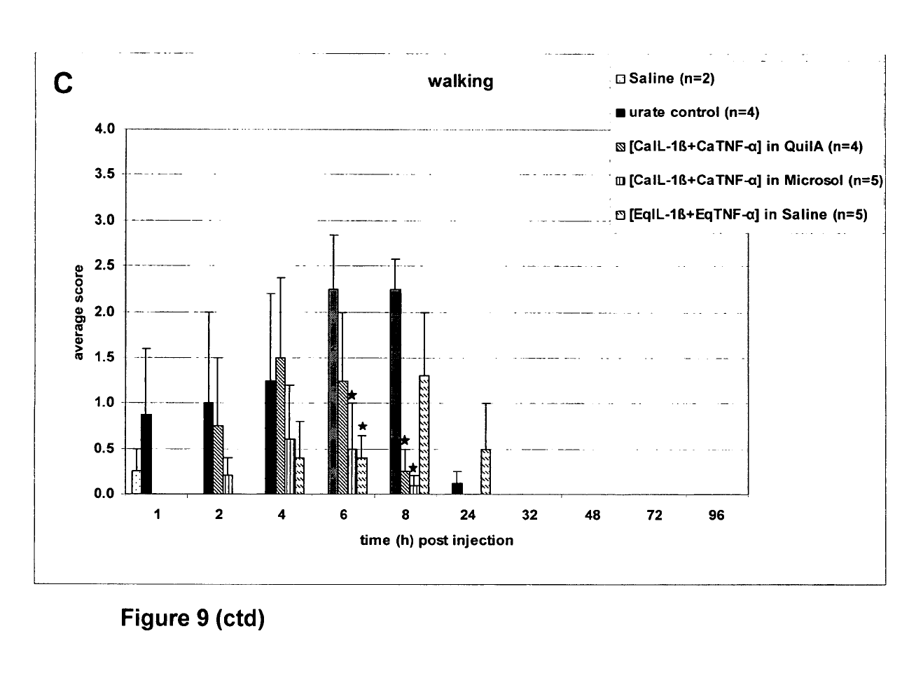

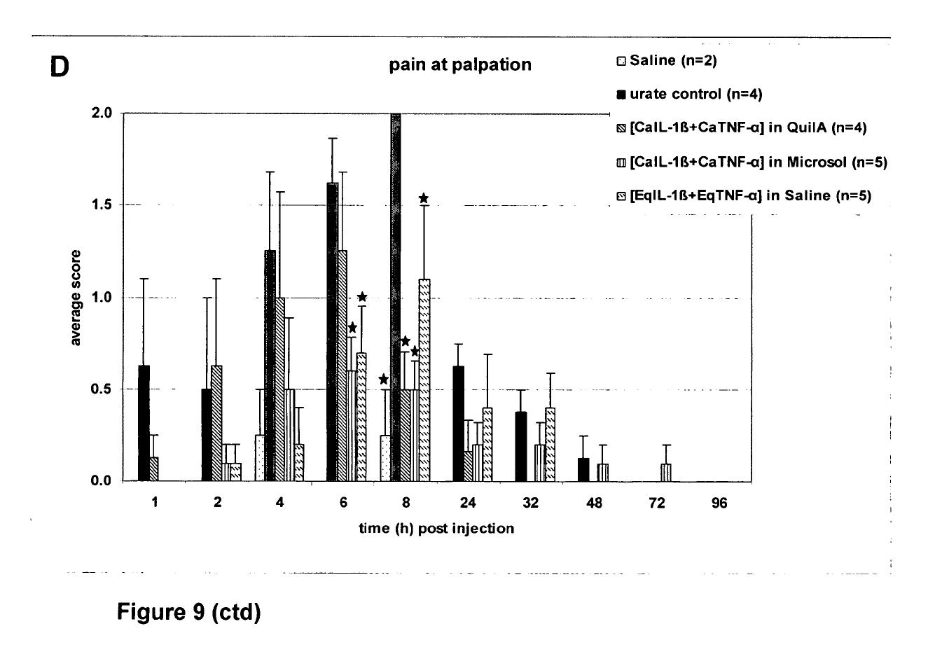

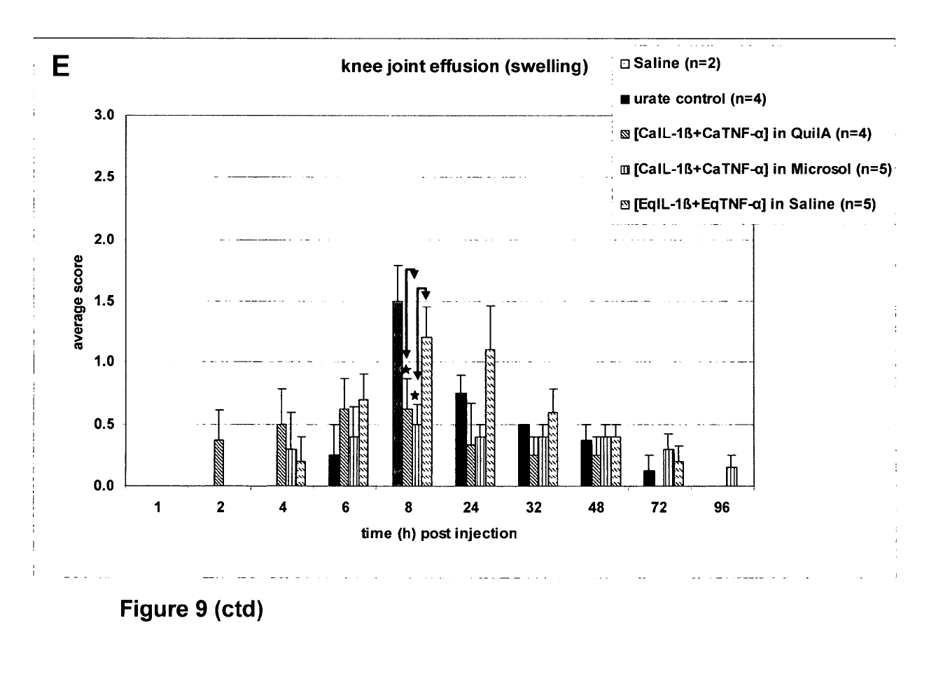

1. A vaccine for the treatment of osteoarthritis in a vertebrate comprising an IL-1β cytokine and a TNF-α cytokines or derivatives thereof, and in association with said IL-1β and said TNF-α or derivatives thereof a part that is non-self with respect to the vertebrate such that the vaccine elicits an immune response in the vertebrate against IL-1β and TNF-α of the vertebrate.

2. The vaccine according to claim 1, wherein said IL-1β and said TNF-α cytokines or derivatives thereof are homologous with respect to the treated vertebrate.

3. The vaccine according to claim 1, wherein the part that is non-self with respect to the vertebrate comprises an antigenic determinant derived from a microorganism.

4. The vaccine according to claim 1 wherein the part that is non-self with respect to the vertebrate comprises an adjuvant.

5. The vaccine according to claim 1 further characterized in that said IL-1β and said TNF-α cytokines or derivatives thereof have a bioactivity that is reduced when compared with the self IL-1β and TNF-α cytokines of the vertebrate.

6-11. (canceled)

12. The vaccine of claim 2, wherein the part that is non-self with respect to the vertebrate comprises an antigenic determinant derived from a microorganism.

13. A method of making the vaccine of claim 1 comprising combining said IL-1β cytokine or derivative thereof and said TNF-α cytokine or derivative thereof with a pharmaceutically acceptable carrier;
wherein in association with said IL-1β cytokine and said TNF-α cytokine or derivatives thereof is a part that is non-self with respect to the vertebrate such that the vaccine elicits an immune response in the vertebrate against IL-1β cytokine and TNF-α cytokine of the vertebrate.

14. The method of claim 13 wherein said IL-1β cytokine or said derivatives thereof is homologous with respect to the treated vertebrate.

15. The method of claim 13 wherein said TNF-α cytokine or said derivatives thereof is homologous with respect to the treated vertebrate.

16. The method of claim 13 wherein the part that is non-self with respect to the vertebrate comprises an antigenic determinant derived from a microorganism.

17. The method of claim 13 wherein said IL-1β cytokine or derivative thereof is associated with an adjuvant to produce the vaccine.

18. The method of claim 13 wherein said TNF-α cytokine or derivative thereof is associated with an adjuvant to produce the vaccine.

19. The method of claim 13, further characterized in that said IL-1β and or derivative thereof has a bioactivity that is reduced when compared with the self IL-1β cytokine of the vertebrate.

20. The method of claim 13, further characterized in that said TNF-α cytokine and or derivative thereof has a bioactivity that is reduced when compared with the self TNF-α cytokine of the vertebrate.

21. A method of treating osteoarthritis in a vertebrate comprising administering the vaccine of claim 12 to the vertebrate.

22. A method of treating osteoarthritis in a vertebrate comprising administering the vaccine of claim 5 to the vertebrate.

23. A method of treating osteoarthritis in a vertebrate comprising administering the vaccine of claim 4 to the vertebrate.

24. A method of treating osteoarthritis in a vertebrate comprising administering the vaccine of claim 3 to the vertebrate.

25. A method of treating osteoarthritis in a vertebrate comprising administering the vaccine of claim 2 to the vertebrate.

26. A method of treating osteoarthritis in a vertebrate comprising administering the vaccine of claim 1 to the vertebrate.
